(19) United States
(12) Patent Application Publication
(10) Pub. No.: US 2018/0031558 A1
(43) Pub. Date: Feb. 1, 2018

(54) BIOMARKERS OF ACUTE LIVER INJURY

(71) Applicant: The Regents of the University of Michigan, Ann Arbor, MI (US)

(72) Inventors: M. Bishr Omary, Ann Arbor, MI (US); Sujith Weerasinghe, Ann Arbor, MI (US)

(21) Appl. No.: 15/727,158

(22) Filed: Oct. 6, 2017

Related U.S. Application Data
(62) Division of application No. 14/713,387, filed on May 15, 2015.
(60) Provisional application No. 61/993,357, filed on May 15, 2014.

(51) Int. Cl. G01N 33/573

(52) U.S. Cl. CPC G01N 33/573; G01N 2333/9015; G01N 2800/085

(57) ABSTRACT

Provided herein are biomarkers of acute liver injury (ALI), which includes acute liver failure, and methods of diagnosing ALI and/or monitoring treatment and prognosis therewith. In particular, serum levels of carbamoyl phosphate synthatase-1 (CPS1) are detected to diagnose and/or monitor treatment and prognosis of ALI.

### CROSS REFERENCE TO RELATED APPLICATIONS

The present application is a divisional of U.S. patent application Ser. No. 14/713,387, filed May 15, 2015, which claims the benefit of U.S. Provisional Patent Application Ser. No. 61/993,357, filed May 15, 2014, each of which is incorporated by reference in its entirety.

### FIELD

Provided herein are biomarkers of acute liver injury (ALI), and methods of diagnosing ALI and/or monitoring treatment therewith. In particular, serum levels of carbamoyl phosphate synthatase-1 (CPS1) are detected to diagnose and/or monitor treatment and recovery of ALI.

### BACKGROUND

A multitude of etiologies can cause or predispose one to liver injury, including toxins (e.g., drugs, alcohol), hepatotropic viruses, metabolic derangements and genetic factors. Such injury may be acute or chronic and involves apoptotic or necrotic cell death. Furthermore, some patients with acute or chronic liver injury may develop severe or progressive disease culminating in liver failure or even death unless rescued by liver transplantation (Malhi et al. Physiol Rev 90: 1165-94, 2010.; herein incorporated by reference in its entirety). There are few biomarkers available that are used in clinical settings to accurately measure the extent of hepatocyte damage. For example, alanine aminotransferase (ALT), which is routinely used as a marker of hepatocellular damage, is expressed in two isoforms (ALT1 and ALT2) and exhibits differential tissue distribution (Lindblom et al. Arch Biochem Biophys 466: 66-77, 2007.; herein incorporated by reference in its entirety). Specifically, ALT1 is expressed in skeletal muscle, kidney and heart in addition to liver; while ALT2 is not expressed in liver but is found in skeletal muscles, heart and pancreas (Id.). However, the standard ALT assay used in clinical settings does not discriminate between the different ALT isoforms or ALT from different tissues (Glinghammar et al. Int J Mol Med 23: 621-31, 2009.; herein incorporated by reference in its entirety). In addition, the caspase-cleaved keratin 18 exposed epitope (M30) and another keratin 18 protein backbone epitope (M65) have been investigated as serum markers of acute (Rutherford et al. Gastroenterology 143: 1237-43, 2012.; herein incorporated by reference in its entirety) and chronic liver disease (Joka et al. Hepatology 55: 455-64, 2012.; herein incorporated by reference in its entirety) and appear to be useful biomarkers. However, they are not specific to liver disease (Greystoke et al. Br J Cancer 107: 1518-24. 2012.; Oyama et al. Clin Exp Med July 24, Epub ahead of print. 2012.; Tas et al. Tumour Biol June 21, Epub ahead of print. 2013.; herein incorporated by reference in their entireties) because of the broad distribution of the abundant cytoskeletal protein, keratin 18, in all simple-type epithelia (Omary et al. J Clin Invest 119: 1794-805, 2009.; herein incorporated by reference in its entirety). Importantly, it is difficult to predict which patients who present with acute liver injury (ALI) are likely to survive without the need for transplantation. The use of the Model for End-Stage Liver Disease and Kings College Criteria remain imperfect (McPhail et al. J Hepatol 53: 492-9. 2010.; Blei A T. Liver Transpl 11: 30-4, 2005.; herein incorporated by reference in their entireties) although a proposed index that includes M30 and clinical markers may offer some advantages (Rutherford et al. Gastroenterology 143: 1237-43, 2012.; herein incorporated by reference in its entirety) recent findings raise some questions whether M30 offers advantage over the standard ALT liver test (Cusi et al. J Hepatol 60:167-74. 2014; herein incorporated by reference in their entireties).

### SUMMARY

In some embodiments, methods of detecting, diagnosing, evaluating, and/or prognosing ALI and/or ALF in a subject are provided. In some embodiments, a method comprises detecting the presence or level of at least one biomarker of ALI and/or ALF in a sample (e.g., plasma, serum, blood, etc.) from the subject. In some embodiments, methods comprise detecting the presence or level of CPS1 in a sample from a subject. In some embodiments, detection of the presence or a particular level of CPS1 in a sample (e.g., plasma, serum, blood, etc.) from the subject (e.g., a level that is altered (e.g., increased) from a control level, a level above a threshold, etc.) is indicative of and/or diagnostic ALI and/or ALF.

In some embodiments, a method comprises detecting the presence or level of CPS1 and at least one, at least two, at least three, at least four, at least five, at least six, at least seven, at least eight, at least nine, or at least ten other biomarkers (e.g., of ALI, or ALF, of chronic liver injury (CLI), of other liver conditions, etc.) in a sample from the subject. In some embodiments, a method comprises detecting the presence or level of CPS1 and the level of one or more of: alanine aminotransferase (ALT), albumin, aspartate transaminase, alkaline phosphatase, total bilirubin, direct bilirubin, gamma glutamyl transpeptidase, 5′ nucleotidase, serum glucose, lactate dehydrogenase. In some embodiments, detection of the presence and/or a particular level of CPS1 and a particular level of one or more of: alanine aminotransferase, albumin, aspartate transaminase, alkaline phosphatase, total bilirubin, direct bilirubin, gamma glutamyl transpeptidase, 5′ nucleotidase, serum glucose, and lactate dehydrogenase in a sample (e.g., plasma, serum, urine, saliva, etc.) from the subject (e.g., a level that is altered (e.g., increased and/or decreased) from a control level of the respective biomarker, a level above or below a threshold, etc.) is indicative or and/or diagnostic for ALI and/or ALF.

In some embodiments, a method of detecting and/or diagnosing ALI and/or ALF in a subject comprises forming a biomarker panel comprising CPS1 and N additional biomarkers. In some embodiments, N biomarkers are selected from alanine aminotransferase, albumin, aspartate transaminase, alkaline phosphatase, total bilirubin, direct bilirubin, gamma glutamyl transpeptidase, 5′ nucleotidase, serum glucose, and lactate dehydrogenase. In some embodiments, N biomarkers comprise ALI, ALF, CLI, or other liver biomarkers not listed herein, but understood in the art. In some embodiments, N is 1 or greater (e.g., >1, >2, >3, >4, >5, >6, >7, >8, >9, >10, >15, >20, >25, >30, >40, >50, or more). In some embodiments, N is 100 or fewer (e.g., <100, <90, <80, <70, <60, <50, <40, <30, <20, <10, <5). In some embodiments, N is 1 to 10. In some embodiments, N is 10 to 20. In some embodiments, N is 5 to 15. In some embodiments, N is 10 to 50. In some embodiments, N is 20 to 100. In some embodiments, N is 4 to 10. In some embodiments, N is 8 to 14. In some embodiments, N is 20 to 30. In some embodiments, N is 1 to 4. In some embodiments, N is 2 to 6. In some embodiments, N is 3 to 7. In some embodiments, at least one of the N biomarker proteins is ALT. In some embodiments, methods further comprise detecting the presence and/or level of each of CPS1 and the N biomarkers of the panel in a sample from the subject.

In some embodiments, a method of detecting and/or diagnosing ALI and/or ALF in a subject comprises forming a biomarker panel comprising CPS1 and X other biomarkers, wherein N of the X biomarkers are selected from alanine aminotransferase, albumin, aspartate transaminase, alkaline phosphatase, total bilirubin, direct bilirubin, gamma glutamyl transpeptidase, 5′ nucleotidase, serum glucose, and lactate dehydrogenase. In some embodiments, X is 100 or fewer (e.g., <90 biomarkers, <80 biomarkers, <70 biomarkers, <60 biomarkers, <50 biomarkers, <40 biomarkers, <30 biomarkers, <20 biomarkers, <15 biomarkers, <10 biomarkers, <5 biomarkers). In some embodiments, X is 1 or greater (e.g., >2, >3, >4, >5, >6, >7, >8, >9, >10, >11 biomarkers, >12 biomarkers, >13 biomarkers, >14 biomarkers, >15 biomarkers, >20 biomarkers, >30 biomarkers, >40 biomarkers, >50 biomarkers). In some embodiments, N is between 0 and 13 (e.g., 0, 1, 2, 3, 4, 5, 6, 7, 8, 9, 10, 11, 12, 13). In some embodiments, at least one of the N biomarker is ALT. In some embodiments, methods further comprise detecting the presence and/or level of each of CPS1 and the X biomarkers of the panel in a sample from the subject.

In some embodiments, methods comprise determining a subject is at risk of ALF and/or ALI by identifying one or more signs or symptoms of ALF or ALI. In some embodiments, signs or symptoms of ALF and/or ALI include: jaundice, abdominal pain, nausea, vomiting, difficulty concentrating, disorientation, confusion, worsening level of consciousness including progression to coma, bleeding diathesis, and/or sleepiness. In some embodiments, methods comprise detecting the presence or level of CPS1 and optionally one or more other biomarkers in a sample from a subject determined to be at risk of ALF and/or ALI based on analysis of symptoms.

In some embodiments, one or more additional steps are taken upon identifying a subject as having ALI and/or ALF. In some embodiments, methods further comprise a subsequent step of treating said subject for ALI and/or ALF (e.g., with medications to address the cause (e.g., poisoning), with liver transplant, to treat complications of ALI/ALF (e.g., excess fluid in the brain, severe bleeding, infections, etc.). In some embodiments, methods further comprise a subsequent step of screening said subject for comorbid infections. In some embodiments, methods further comprise a subsequent step of additional diagnostic steps (e.g., imaging (e.g., x-ray, MRI, ultrasound) to identify liver damage, biopsy (e.g., transjugular biopsy), detection of additional biomarkers, etc.). In some embodiments, methods further comprise generating a report diagnosing said subject as having ALI and/or ALF or indicating a likelihood of said subject having ALI and/or ALF.

In some embodiments described herein, a biomarker is a protein biomarker. In any of the embodiments described herein, the method may comprise contacting biomarkers of the sample from the subject with a set of biomarker detection reagents. In any of the embodiments described herein, the method may comprise contacting biomarkers of the sample from the subject with a set of biomarker capture reagents, wherein each biomarker capture reagent of the set of biomarker capture reagents specifically binds to a biomarker being detected. In some embodiments, each biomarker capture reagent of the set of biomarker capture reagents specifically binds to a different biomarker being detected. In some embodiments, each biomarker capture reagent may be an antibody or an aptamer.

In certain embodiments, the sample may be a blood sample, plasma sample, serum sample, etc. In some embodiments, the sample (e.g., blood, serum, plasma, etc.) is processed (e.g., diluted, concentrated, filtered, fractionated, etc.). In some embodiments, the sample (e.g., blood, serum, plasma, etc.) is unprocessed.

In some embodiments, methods of monitoring progression of ALI and/or ALF in a subject are provided. In some embodiments, a method comprises detecting the presence or level of CPS1 and optionally one or more additional biomarkers in a sample from the subject at a first time point. In some embodiments, the method further comprises detecting the presence or level of CPS1 and optionally one or more additional biomarkers in a sample from the subject at a second and additional time points. In some embodiments, ALI and/or ALF is worsening if the level of CPS1 is higher at the second time point than at the first time point. In some embodiments, ALI and/or ALF is worsening if the level of CPS1 and one or more of the additional biomarkers in a sample are further removed from a control value, control range, and/or threshold than at the first time point. In some embodiments, ALI and/or ALF is improving if the level of CPS1 is lower at the second or subsequent time points than at the first or prior time points. In some embodiments, ALI and/or ALF is improving if the level of CPS1 and one or more of the additional biomarkers in a sample from the subject are less removed from a control value, control range, and/or threshold than at the first time point. In some embodiments, first and second time points are separated by at least 1 hour, 1 day, at least 2 days, at least 4 days, at least 1 week, at least 2 weeks, at least 1 month, at least 2 months, at least 3 months, at least 4 months, at least 6 months, or by 1 year or more. In some embodiments, first and second time points are separated by no more than 1 day, 1 week, 2 weeks, 1 month, 2 months, 3 months, 4 months, 6 months, or 1 year. In some embodiments, methods further comprise detecting the presence or level of CPS1 and optionally one or more additional biomarkers in a sample from the subject at a third time point, a fourth time point, a fifth time point, a sixth time point, a seventh time point, an eighth time point, etc. In some embodiments, levels of CPS1 and optionally other biomarkers are monitored over several time points to monitor the progression (severity over time) of the ALI and/or ALF. In some embodiments, subsequent time points are separated by at least 1 hour, 1 day, at least 2 days, at least 4 days, at least 1 week, at least 2 weeks, at least 1 month, at least 2 months, at least 3 months, at least 4 months, at least 6 months, or by 1 year or more. In some embodiments, subsequent time points are separated by no more than 1 day, 1 week, 2 weeks, 1 month, 2 months, 3 months, 4 months, 6 months, or 1 year.

In some embodiments, CPS1 and one or more additional biomarkers are detected at a first time point to establish that the subject has suffered ALI and/or has ALF, and CPS1 is monitored at one or more additional timepoints to monitor progression thereof In some embodiments, reduction of CPS1 levels in subsequent time points indicates that ALF is subsiding (e.g., naturally or in response to treatment or therapy) which can have positive prognostic implications and allow downgrading the critical nature of the ALF.

In some embodiments, methods of monitoring treatment of ALI and/or ALF in a subject are provided. In some embodiments, a method comprises: (a) detecting the presence or level of CPS1 and optionally one or more additional biomarkers in a sample from the subject at a first time point; (b) administering a treatment for ALF and/or ALI to the subject; and (c) measuring the presence or level of CPS1 and optionally one or more additional biomarkers in a sample from the subject at a first time point. In some embodiments, treatment is ineffective if the levels at the second timepoint are unchanged or further removed from a control value, control range, and/or threshold than at the first time point. In some embodiments, treatment is effective if the levels are less removed from a control value, control range, and/or threshold than at the first time point. In some embodiments, first and second time points are separated by at least 1 hour, 1 day, at least 2 days, at least 4 days, at least 1 week, at least 2 weeks, at least 1 month, at least 2 months, at least 3 months, at least 4 months, at least 6 months, or by 1 year or more. In some embodiments, first and second time points are separated by no more than 1 day, 1 week, 2 weeks, 1 month, 2 months, 3 months, 4 months, 6 months, or 1 year. In some embodiments, if treatment is determined to be ineffective, an alternative course of treatment is administered. In some embodiments, methods further comprise detecting the presence or level of CPS1 and optionally one or more additional biomarkers in a sample from the subject at a third time point, a fourth time point, a fifth time point, a sixth time point, a seventh time point, an eighth time point, etc. while treating the subject for ALI/ALF. In some embodiments, levels of CPS1 and optionally other biomarkers are monitored over several time points to monitor the treatment. In some embodiments, subsequent time points are separated by at least 1 hour, 1 day, at least 2 days, at least 4 days, at least 1 week, at least 2 weeks, at least 1 month, at least 2 months, at least 3 months, at least 4 months, at least 6 months, or by 1 year or more. In some embodiments, subsequent time points are separated by no more than 1 day, 1 week, 2 weeks, 1 month, 2 months, 3 months, 4 months, 6 months, or 1 year.

In some embodiments, kits are provided. In some embodiments, a kit comprises a reagent (e.g., detection reagents, capture reagents, etc.) specific for CPS1. In some embodiments, a kit comprises a reagent (e.g., detection reagents, capture reagents, etc.) specific for CPS1 and at least one, at least two, at least three, at least four, at least five, at least six, at least seven, at least eight, or at least nine reagents specific for other biomarkers (e.g., ALI biomarkers, ALF biomarkers, CLF biomarkers, liver biomarkers, etc.). In some embodiments, other biomarkers are selected from alanine aminotransferase, albumin, aspartate transaminase, alkaline phosphatase, total bilirubin, direct bilirubin, gamma glutamyl transpeptidase, 5′ nucleotidase, serum glucose, and lactate dehydrogenase. In some embodiments, a reagent is an antibody or aptamer. In some embodiments, each reagent specifically binds to a different target biomarker. In some embodiments, a kit comprises reagents (e.g., antibody, aptamer, tagged, untagged, etc.) that specifically binds CPS1; optionally one or more reagents that specifically bind one or more of alanine aminotransferase, albumin, aspartate transaminase, alkaline phosphatase, total bilirubin, direct bilirubin, gamma glutamyl transpeptidase, 5′ nucleotidase, serum glucose, and lactate dehydrogenase; optionally one or more reagents that specifically bind one or more other ALF, ALI, CLI, or liver biomarkers; and optionally one or more other non-liver biomarkers.

In some embodiments, a kit comprises a reagent that specifically binds CPS1 and X additional reagents (e.g., detection reagents, capture reagents, etc.) specific for other biomarkers, wherein N reagents specifically bind to a biomarker protein selected from alanine aminotransferase, albumin, aspartate transaminase, alkaline phosphatase, total bilirubin, direct bilirubin, gamma glutamyl transpeptidase, 5′ nucleotidase, serum glucose, and lactate dehydrogenase. In some embodiments, X is less than 100 (e.g., <90, <80, <70, <60, <50, <40, <30, <20, <15). In some embodiments, X is 1 or more (e.g., >1, >2, >3, >4, >5, >6, >7, >8, >9, >10, >11, >12, >13, >14, >15, >20, >30, >40, >50. In some embodiments, N is 1 to 13 (1, 2, 3, 4, 5, 6, 7, 8, 9, 10, 11, 12, 13). In some embodiments, at least one of the N biomarker proteins is selected from ALT.

### BRIEF DESCRIPTION OF THE DRAWINGS

FIG. 1. Primary mouse hepatocytes release CPS1 during FasL induced apoptotic injury. A: Coomassie stain of concentrated culture media collected from FasL treated and untreated hepatocytes. FasL treated hepatocytes showed many released proteins into the culture media as compared to untreated cells. Mass spectrometry identified the five most intense protein bands (1-5) as indicated. B: Left panel: CPS1 Immunoblot of culture media confirms the mass spectrometry prediction of band 1; Right panel: Immunoblot analysis of total cell lysates from FasL treated and untreated hepatocytes. The antibodies were directed to: caspases 3 and 7, actin and keratin 18 (K18) which recognizes K18 and its apoptotic fragment (arrow). An equal amount of the concentrated culture media and total cell lysate was loaded.

FIG. 2. Primary hepatocytes release CPS1 in response to difference types of liver injury. A: Primary mouse hepatocytes were cultured in the presence of hypoosmotic media (200 mOsm/L), FasL (0.5 μg/ml), and acetaminophen (APAP, 1 mM). The concentrated cell culture media and total cell lysates were used for immunoblotting with antibodies to the indicated proteins. B: After each treatment as in panel A, the viable cell count was determined using trypan blue staining after trypsin treatment. Each bar represents the mean of 10 to 15 fields. Note that significant cell deaths occurred during FasL and APAP treatment, however, no detectable cell death occurs with cells cultured in the presence of hypoosmotic media. C: Mouse hepatocytes were challenged with FasL, APAP and hypoosmosis as in Panel A. Cells were fixed and immunostained with CPS1 (red) and counter stained with DAPI (blue). Arrows highlight areas with less dense CPS1 staining. Scale bar=20 μm.

FIG. 3. Hepatocyte selective CPS1 expression in liver. A: Liver sections form normal mice were fixed in methanol (−20° C., 10 min) and immunostained with antibodies to CPS1 (red) and hepatocyte specific marker, keratin 18 (green). Hepatocytes show the expression of CPS1 (merged, panel c). B: Sections same liver were immunostained with CPS1 (red) and ductal cell specific marker keratin 19 (green). Ductal cells do not express CPS1 (merged, panel c). DAPI staining shows nuclei (blue). Scale=50 μm.

FIG. 4. CPS1 is released into serum during FasL and APAP induced mouse liver injury. Mice were injected with FasL (0.15 μg/g) and APAP (700 mg/Kg) and sacrificed after 4 h and 8 h, respectively. Liver damage was analyzed serologically, biochemically and histologically. Panel A: Immunoblots of serum CPS1, HMGB1 and caspase activation using total liver lysate. Coomassie stain of serum IgG heavy chain (just above the 50 kDa prominent species) and IgG light chain (25 kDa), and an actin blot of total liver lysates are included as loading controls. Serum ALT of the individual mice is also indicated (bottom of panel). Panel B: Hematoxylin and eosin stained liver sections. FasL treatment (b) shows severe hemorrhage formation (arrows), while APAP (d) induces necrotic cell death (arrows) as compared to control treatments using vehicles (a and c). Scale bar=40 μm.

FIG. 5. Kinetics of CPS1 release in apoptotic mouse liver injury. Mice were injected with FasL (0.15 μg/g), and sacrificed at the indicated time points followed by serum collection. Panel A: Time dependent serum CPS1 release into blood. Serum CPS1 was detected as early as 2 h after FasL injection and CPS1 levels increased with time. Parallel to CPS1 levels, serum ALT levels also increase in a time-dependent manner after FasL injection. Coomassie stain of serum IgG is included to show equal protein loading. Panel B: Livers were isolated from the same animals used in panel A and homogenized in SDS-containing sample buffer to obtain a total liver homogenate. The liver lysates were analyzed by immunoblotting using antibodies to the indicated antigens. The keratin 18 (K18) blot is included as a loading control. Collectively these findings indicate that CPS1 release is commensurate with onset of apoptosis. Panel C: Serum and plasma are equally suited to detect CPS1 release into the circulation. Three mice were injected with FasL (0.15 μg/g) and blood was collected from the sacrificed mice after 4 h. A separate mouse (Con) was injected with saline and used as a control. Blood from each mouse was divided and processed to obtain serum and plasma. CPS1 was equally detected in serum and plasma of all FasL mice. Coomassie stain of serum IgG is included as loading control. The serum ALT of mice is indicated. S, Serum; P, Plasma.

FIG. 6. CPS1 has a short half-life in mouse serum. In order to determine the half-life of CPS1 in mouse serum, mice (five total) were injected with FasL (0.075 μg/g) and blood was collected from the same animal via tail vain at the indicated time points. Sera were used to determine serum ALT, or mixed with 2× SDS sample buffer to measure circulating serum CPS1 by western blot analysis. CPS1 pixel intensity (CPS1 OD) at each time point was determined by densitometry scanning. Panels A-D: Immunoblots of time-dependent serum CPS1 levels of four individual mice are shown. CPS1 OD and serum ALT values at each time point are indicated. A Coomassie stain of serum is included as loading control. To determine the half-life of serum CPS1, the OD values of 24 h to 32 h of all five mice were used. Assuming an exponential decay, half-life of CPS1 for each time interval was calculated using the equation; Ending amount=Beginning amount/2n; where n=number of half-lives. The average half-life of CPS1 in mouse serum was determined to be 126±9 min (mean±standard error of mean).

FIG. 7. CPS1 is detected in sera of human patients with ALF. The presence of CPS1 in serum of human patients with different forms of ALF was tested using serum collected from day 1 up to day 7 of hospitalization. Panel A: Analysis of CPS1 in serial serum samples collected from a patient with APAP. Sera were separated on duplicate gels by SDS-PAGE, with one gel stained by Coomassie blue to visualize the immunoglobulin heavy (H) and light (L) chains. The second gel was transferred to a membrane for subsequent immunoblotting using anti-CPS1 antibody. ALT values from the same serum samples are also included. (Panels B and C) Serial serum samples from a patient with ischemic hepatitis and with Wilson disease were analyzed as in Panel A.

FIG. 8. CPS1 turns over more rapidly than ALT in patients with APAP-related ALF. Panels A-C: Immunoblots of serum CPS1 from patients with APAP-related ALF (sera were collected on days 1 to 7 of hospitalization; days 4-7 were not available for the patient in Panel C). The Coomassie stain of serum indicates equal loading (H, heavy chain; L, light chain). ALT values from the same serum samples are also shown. D: % CPS1 and % ALT values as compared with day 1 values are plotted for each of the days. CPS1 relative levels were obtained by densitometry scanning. Decay of serum CPS1 was much faster than that of ALT. Serial serum samples from four APAP patients who had up to 7 days of serial serum samples (three shown in Panels A-C, and one shown in FIG. 7, Panel A) were used for quantification.

FIG. 9. Serum CPS1 in chronic liver injury. Panel A: The presence of serum CPS1 and HMGB1 was tested in animals exposed to the hepatotoxin DDC for 10 days, 15 days, 6 weeks and 3 months. Corresponding serum ALT values are also included. Coomassie stain of serum IgG showed equal serum protein loading. Each lane corresponds to serum from separate mice. Two mice for each time point were shown except the control. Panel B: Hematoxylin and eosin staining of livers from control and DDC-administered mice. Scale bar=40 μm. Panels C and D: Human sera from patients with chronic hepatitis B virus infection (HBV), chronic hepatitis C infection (HCV), or controls were analyzed for the presence of CPS1 and HMGB1. Serum from APAP and from control donors with normal ALT were used as positive and negative controls, respectively. Coomassie stain of serum showed similar protein loading. Each lane represents separate patients or donors.

FIG. 10. Model for CPS1 and ALT release in the context of acute liver injury; although the present invention is not limited to any particular mechanism of action and an understanding of the mechanism of action is not necessary to practice the present invention. The mitochondrial matrix protein CPS1 and the cytoplasmic ALT are released to the blood circulation upon ALI. This occurs in the context of apoptotic (e.g., via FasL) or necrotic (e.g., via APAP) liver injury. During recovery from liver injury, serum CPS1 levels drop rapidly while the serum ALT decline is more gradual.

### Definitions

As used herein, “acute liver failure” is the rapid (e.g., <7 days) appearance of severe symptoms and complications of liver disease. Acute liver failure is typically triggered by an instigating “acute liver injury insult” Exemplary events that cause acute liver injuries and can lead to acute liver failure include acetaminophen overdose, use of certain medications (e.g., antibiotics, nonsteroidal anti-inflammatory drugs, anticonvulsants, etc.), the use of herbal supplements (e.g., kava, ephedra, skullcap, pennyroyal, etc.), hepatitis A, hepatitis B, hepatitis E, Epstein-Barr virus, cytomegalovirus, herpes simplex virus, toxins, autoimmune disease (e.g., autoimmune hepatitis), vascular disease (e.g., Budd-Chiari syndrome), metabolic disease (e.g., Wilson's disease), and cancer.

As used herein, “chronic liver disease” or “chronic liver failure” refers to long-term disease processes of the liver that involve progressive destruction and regeneration of the liver parenchyma leading to fibrosis and cirrhosis.

“Biological sample”, “sample”, and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), sputum, tears, mucus, nasal washes, nasal aspirate, breath, urine, semen, saliva, peritoneal washings, ascites, cystic fluid, meningeal fluid, amniotic fluid, glandular fluid, lymph fluid, nipple aspirate, bronchial aspirate (e.g., bronchoalveolar lavage), bronchial brushing, synovial fluid, joint aspirate, organ secretions, cells, a cellular extract, and cerebrospinal fluid. This also includes experimentally separated fractions of all of the preceding. For example, a blood sample can be fractionated into serum, plasma, or into fractions containing particular types of blood cells, such as red blood cells or white blood cells (leukocytes). In some embodiments, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas, and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual.

Further, in some embodiments, a biological sample may be derived by taking biological samples from a number of individuals and pooling them, or pooling an aliquot of each individual's biological sample. The pooled sample may be treated as described herein for a sample from a single individual, and, for example, if ALF/ALI is detected in the pooled sample, then each individual biological sample can be re-tested to identify the affected individual(s).

“Target”, “target molecule”, and “analyte” are used interchangeably herein to refer to any molecule of interest that may be present in a biological sample. A “molecule of interest” includes any minor variation of a particular molecule, such as, in the case of a protein, for example, minor variations in amino acid sequence, or gain or loss of modifications including disulfide bond formation, glycosylation, lipidation, acetylation, phosphorylation, or any other manipulation or modification, such as conjugation with a labeling component, which does not substantially alter the identity of the molecule/protein. A “target molecule”, “target”, or “analyte” refers to a set of copies of one type or species of molecule or multi-molecular structure. “Target molecules”, “targets”, and “analytes” refer to more than one type or species of molecule or multi-molecular structure. Exemplary target molecules include proteins, polypeptides, nucleic acids, carbohydrates, lipids, polysaccharides, glycoproteins, hormones, receptors, antigens, antibodies, affybodies, antibody mimics, viruses, pathogens, toxic substances, substrates, metabolites, transition state analogs, cofactors, inhibitors, drugs, dyes, nutrients, growth factors, cells, tissues, and any fragment or portion of any of the foregoing. In some embodiments, a target molecule is a protein, in which case the target molecule may be referred to as a “target protein.”

As used herein, a “capture agent” or “capture reagent” refers to a molecule that binds specifically to a biomarker. A “target protein capture reagent” refers to a molecule that binds specifically to a target protein. Nonlimiting exemplary capture reagents include aptamers, antibodies, adnectins, ankyrins, other antibody mimetics and other protein scaffolds, autoantibodies, chimeras, small molecules, nucleic acids, lectins, ligand-binding receptors, imprinted polymers, avimers, peptidomimetics, hormone receptors, cytokine receptors, synthetic receptors, and modifications and fragments of any of the aforementioned capture reagents.

The term “antibody” refers to full-length antibodies of any species and fragments and derivatives of such antibodies, including Fab fragments, F(ab′)2 fragments, single chain antibodies, Fv fragments, and single chain Fv fragments. The term “antibody” also refers to synthetically-derived antibodies, such as phage display-derived antibodies and fragments, affybodies, nanobodies, etc.

As used herein, “marker” and “biomarker” are used interchangeably to refer to a target molecule that indicates or is a sign of a normal or abnormal process in an individual or of a disease or other condition in an individual. More specifically, a “marker” or “biomarker” is an anatomic, physiologic, biochemical, or molecular parameter associated with the presence of a specific physiological state or process, whether normal or abnormal, and, if abnormal, whether chronic or acute. Biomarkers are detectable and measurable by a variety of methods including laboratory assays and medical imaging. In some embodiments, a biomarker is a target protein.

As used herein, “biomarker level” and “level” refer to a measurement that is made using any analytical method for detecting the biomarker in a biological sample and that indicates the presence, absence, absolute amount or concentration, relative amount or concentration, titer, a level, an expression level, a ratio of measured levels, or the like, of, for, or corresponding to the biomarker in the biological sample. The exact nature of the “level” depends on the specific design and components of the particular analytical method employed to detect the biomarker.

A “control level” of a target molecule refers to the level of the target molecule in the same sample type from an individual that does not have the disease or condition (e.g., ALF, ALI, etc.). A “control level” of a target molecule need not be determined each time the present methods are carried out, and may be a previously determined level that is used as a reference or threshold to determine whether the level in a particular sample is higher or lower than a normal level. In some embodiments, a control level in a method described herein is the level that has been observed in one or more subjects without ALF/ALI. In some embodiments, a control level in a method described herein is the average or mean level, optionally plus or minus a statistical variation, that has been observed in a plurality of subjects without ALF/ALI.

A “threshold level” of a target molecule refers to the level beyond which (e.g., above or below, depending upon the biomarker) is indicative of or diagnostic for a particular disease or condition. A “threshold level” of a target molecule need not be determined each time the present methods are carried out, and may be a previously determined level that is used as a reference or threshold to determine whether the level in a particular sample is higher or lower than a normal level. In some embodiments, a subject with a biomarker level beyond (e.g., above or below, depending upon the biomarker) a threshold level has a statistically significant likelihood (e.g., 80% confidence, 85% confidence, 90% confidence, 95% confidence, 98% confidence, 99% confidence, 99.9% confidence, etc.) of having ALF/ALI.

As used herein, “individual” and “subject” are used interchangeably to refer to a test subject or patient. The individual can be a mammal or a non-mammal. In various embodiments, the individual is a mammal. A mammalian individual can be a human or non-human. In various embodiments, the individual is a human. A healthy or normal individual is an individual in which the disease or condition of interest (e.g., ALF/ALI) is not detectable by conventional diagnostic methods.

“Diagnose”, “diagnosing”, “diagnosis”, and variations thereof refer to the detection, determination, or recognition of a health status or condition of an individual on the basis of one or more signs, symptoms, data, or other information pertaining to that individual. The health status of an individual can be diagnosed as healthy/normal (e.g., a diagnosis of the absence of a disease or condition) or diagnosed as ill/abnormal (e.g., a diagnosis of the presence, or an assessment of the characteristics, of a disease or condition). The terms “diagnose”, “diagnosing”, “diagnosis”, etc., encompass, with respect to a particular disease or condition, the initial detection of the disease; the characterization or classification of the disease; the detection of the progression, remission, or recurrence of the disease; and the detection of disease response after the administration of a treatment or therapy to the individual.

“Prognose”, “prognosing”, “prognosis”, and variations thereof refer to the prediction of a future course of a disease or condition in an individual who has the disease or condition (e.g., predicting patient survival, predicting the need for organ transplant, etc.), and such terms encompass the evaluation of disease response after the administration of a treatment or therapy to the individual.

“Evaluate”, “evaluating”, “evaluation”, and variations thereof encompass both “diagnose” and “prognose” and also encompass determinations or predictions about the future course of a disease or condition in an individual who does not have the disease as well as determinations or predictions regarding the likelihood that a disease or condition will recur in an individual who apparently has been cured of the disease. The term “evaluate” also encompasses assessing an individual's response to a therapy, such as, for example, predicting whether an individual is likely to respond favorably to a therapeutic agent or is unlikely to respond to a therapeutic agent (or will experience toxic or other undesirable side effects, for example), selecting a therapeutic agent for administration to an individual, or monitoring or determining an individual's response to a therapy that has been administered to the individual. Thus, “evaluating” ALF/ALI can include, for example, any of the following: diagnosing a subject as having experienced ALI, diagnosing a subject as suffering from ALF, determining a subject should undergo further testing (e.g., biopsy); prognosing the future course of ALI/ALF in an individual; determining whether a treatment being administered is effective in the individual; determining whether an individual will require organ transplant (e.g., liver transplant), determining whether an individual will recover without treatment (e.g., further treatment; or selecting a treatment to administer to an individual based upon a determination of the biomarker levels derived from the individual's biological sample.

As used herein, “detecting” or “determining” with respect to a biomarker level includes the use of both the instrument used (if used) to observe and record a signal corresponding to a biomarker level and the material/s required to generate that signal. In various embodiments, the level is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical or biochemical detection methods, nuclear magnetic resonance, quantum dots, and the like.

“Solid support” refers herein to any substrate having a surface to which molecules may be attached, directly or indirectly, through either covalent or non-covalent bonds. A “solid support” can have a variety of physical formats, which can include, for example, a membrane; a chip (e.g., a protein chip); a slide (e.g., a glass slide or coverslip); a column; a hollow, solid, semi-solid, pore- or cavity- containing particle, such as, for example, a bead; a gel; a fiber, including a fiber optic material; a matrix; and a sample receptacle. Exemplary sample receptacles include sample wells, tubes, capillaries, vials, and any other vessel, groove or indentation capable of holding a sample. A sample receptacle can be contained on a multi-sample platform, such as a microtiter plate, slide, microfluidics device, and the like. A support can be composed of a natural or synthetic material, an organic or inorganic material. The composition of the solid support on which capture reagents are attached generally depends on the method of attachment (e.g., covalent attachment). Other exemplary receptacles include microdroplets and microfluidic controlled or bulk oil/aqueous emulsions within which assays and related manipulations can occur. Suitable solid supports include, for example, plastics, resins, polysaccharides, silica or silica-based materials, functionalized glass, modified silicon, carbon, metals, inorganic glasses, membranes, nylon, natural fibers (such as, for example, silk, wool and cotton), polymers, and the like. The material composing the solid support can include reactive groups such as, for example, carboxy, amino, or hydroxyl groups, which are used for attachment of the capture reagents. Polymeric solid supports can include, e.g., polystyrene, polyethylene glycol tetraphthalate, polyvinyl acetate, polyvinyl chloride, polyvinyl pyrrolidone, polyacrylonitrile, polymethyl methacrylate, polytetrafluoroethylene, butyl rubber, styrenebutadiene rubber, natural rubber, polyethylene, polypropylene, (poly)tetrafluoroethylene, (poly)vinylidenefluoride, polycarbonate, and polymethylpentene. Suitable solid support particles that can be used include, e.g., encoded particles, such as Luminex®-type encoded particles, magnetic particles, and glass particles.

As used herein, the term “prothrombin time” refers to a laboratory measurement of the time required for the liquid portion (plasma) of blood to clot. “Prothrombin ratio” refers to a value that is derived by dividing prothrombin time by the result for a control plasma sample. “International normalized ratio” refers to the prothrombin ration raised to the power of the “international sensitivity index” value for the control plasma sample.

### DETAILED DESCRIPTION

Provided herein are biomarkers of acute liver injury (ALI), and methods of diagnosing ALI and/or monitoring treatment and/or prognosis therewith. In particular, serum levels of carbamoyl phosphate synthatase-1 (CPS1) are detected to diagnose and/or monitor treatment and/or disease course of ALI.

Carbamoyl phosphate synthatase-1 (CPS1) is the most abundant mitrochondrial matrix protein, and functions in the urea cycle. CPS1 is preferentially expressed in the liver (selectively in hepatocytes) with much lower expression in the intestine and kidney. Carbamoyl phosphate synthetase-1 (CPS1) is the most abundant protein in liver mitochondria; and it accounts for nearly 20% of the matrix protein mass but it is expressed in much lower levels in intestine, kidney and fibroblasts (Hu et al. Theranostics 4: 215-228, 2014.; Martinez et al. Mol Genet Metab 101: 311-23, 2010.; Rapp et al. Eur J Pediatr 160: 283-7, 2001.; herein incorporated by reference in their entireties). In the mitochondrial matrix, CPS1 catalyzes the conversion of ammonia and bicarbonate into carbamoyl phosphate which is the first and rate limiting step in the urea cycle (Martinez et al. Mol Genet Metab 101: 311-23, 2010.; herein incorporated by reference in its entirety).

Experiments were conducted during development of embodiments of the present invention using an unbiased approach to identify proteins that are released into culture media upon exposure of primary mouse hepatocytes to Fas ligand (FasL) which induces hepatocyte cell death. CPS1 was identified as a major protein released into the media at a level similar to that seen with albumin. The extent of CPS1 release in response to mouse and human liver injury was then assessed. Experiments demonstrate that CPS1 is a robust biomarker in acute liver injury that is rapidly cleared from serum.

Experiments conducted during development of embodiments of the present invention demonstrate that CPS1 can be detected in human serum of patients with ALI [e.g., due to acetaminophen (APAP) over dose, ischemia and Wilson disease]. The experiments demonstrated that CPS1 disappears rapidly from the serum during patient recovery while ALT, the gold standard liver injury marker, remains elevated. These studies using human serum are supported by extensive experiments conducted in mouse experimental acute liver injury and in cultured mouse hepatocytes subjected to various modalities of injury.

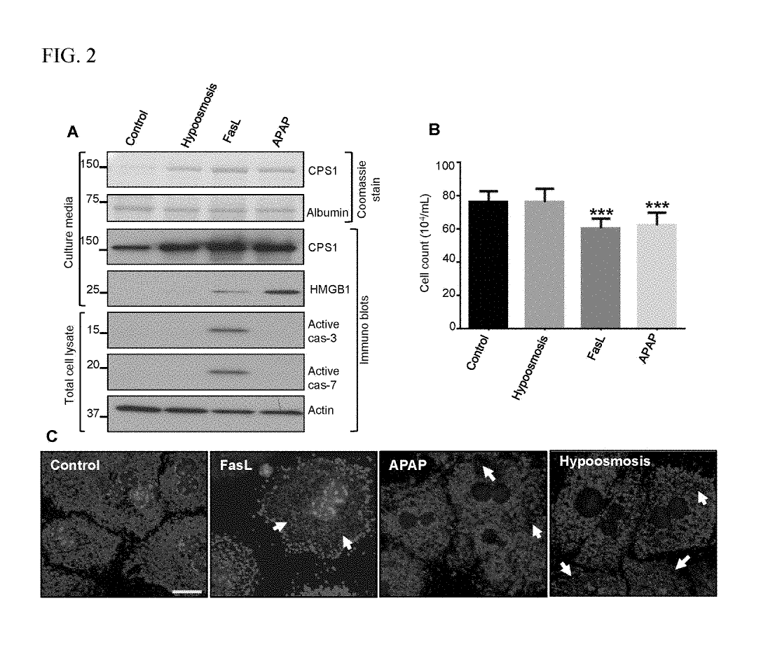

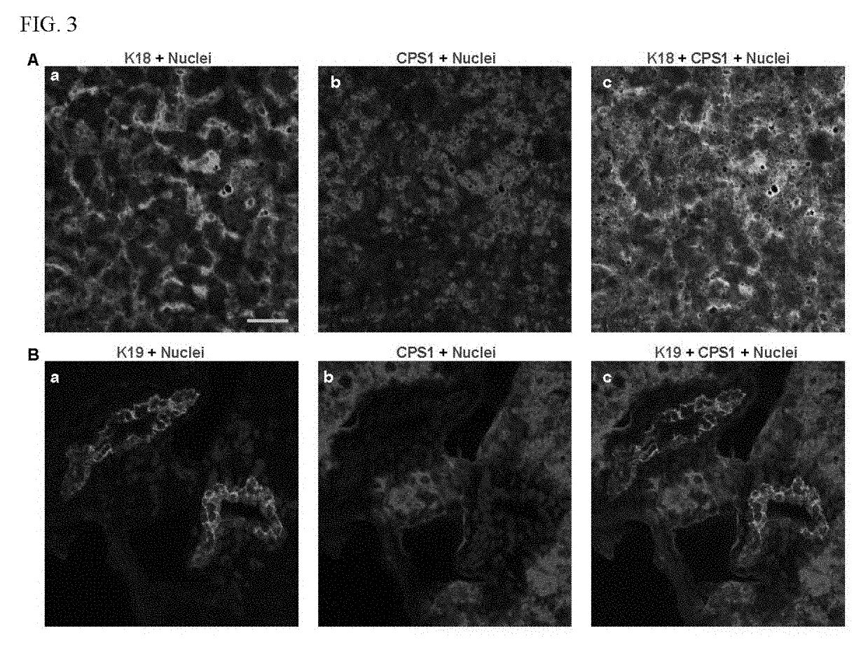

Ex vivo and in vivo mouse liver injury models provide strong evidence that the hepatocyte-selective and most abundant mitochondrial matrix-protein, CPS1, is a marker for apoptotic and necrotic forms of hepatocyte death and injury. The marked release of CPS1 during hypoosmotic stress (FIG. 2), without evidence of cell death per se, suggests that such release may represent a state of organelle and cell leakiness that does not necessarily require cell death; although the present invention is not limited to any particular mechanism of action and an understanding of the mechanism of action is not necessary to practice the present invention. It is contemplated that the mechanism of release of CPS1 from the hepatocyte may be multifactorial, and may reflect organelle spillage and cell rupture (e.g., during necrosis) or a nonclassical release that is vesicle (e.g., exosome or microparticle) or nonvesicle related. In addition, prominent release of CPS1 by primary hepatocytes in response to hypoosmosis, apoptosis (e.g., via FasL) and necrosis (e.g., via APAP) may be associated with distinct mechanism of release depending on the injury model. This is supported by the observed different combinations of caspase activation and HMGB1 (High-Mobility Group Protein B1, a marker of cell necrosis) release associated with of each hepatocyte injury model (FIG. 2A). The abundance of CPS1, and relative selective expression in hepatocytes (FIG. 3) makes it a very attractive candidate as an acute liver injury biomarker. Liver appears to be the dominant human organ for CPS1 expression, with the small intestine having the next highest expression level (Neill et al. Mol Genet Metab 97: 121-7, 2009.; herein incorporated by reference in its entirety). For example, mRNA Ct values (using qRT-PCR) for CPS1 in human liver were 22.85 while those for small intestine were 25.86 (Id.).

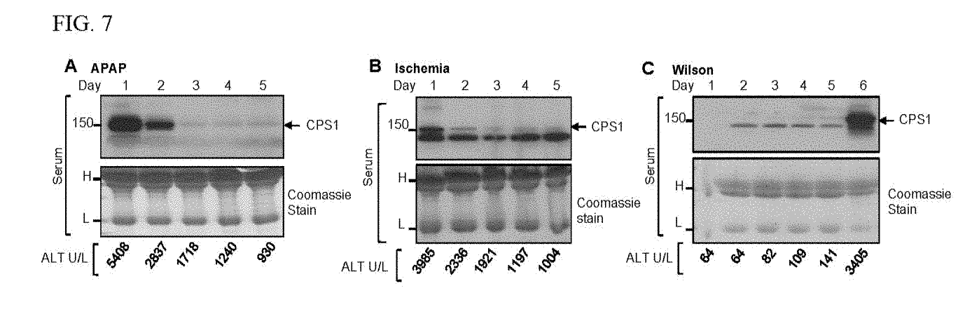

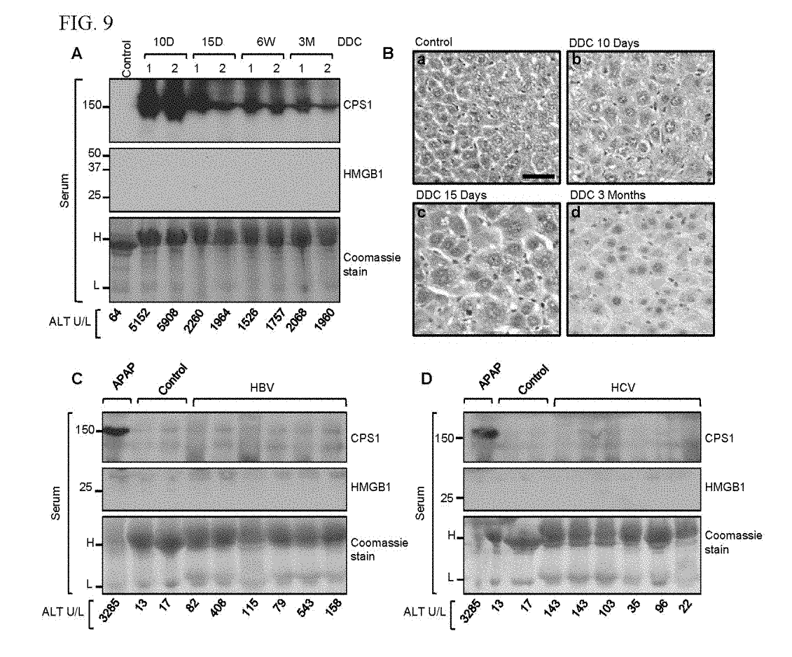

CPS1 has utility as a biomarker of acute liver injury, and is capable of discriminating such injury from chronic liver injuries (e.g., in both mice and humans). A dramatic increase in serum CPS1 was observed in human ALI due to APAP, ischemia and Wilson disease (FIG. 7). In contrast, CPS1 was not detected in patients with chronic viral hepatitis (FIG. 9). Furthermore, serum CPS1 levels decreased in the DDC mouse toxicity model as exposure moved from acute to subacute and chronic (FIG. 9).

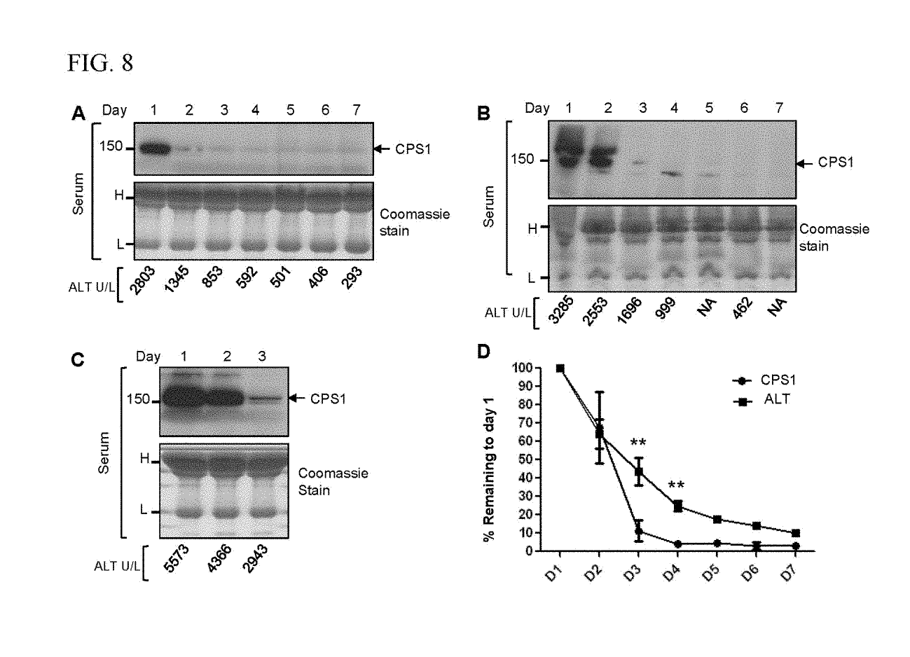

A recent report demonstrated that while CPS1 can be detected in mouse serum in response to ALI, CPS1 could not be detected in human serum of subjects with ALI (Hu et al. Theranostics 4: 215-228, 2014.; herein incorporated by reference in its entirety). However, experiments conducted during development of embodiments of the present invention clearly demonstrate detectable increase of CPS1 in human serum reflecting the severity of liver injury. Experiments conducted during development of embodiments of the present invention indicate that CPS1 is detectable in serum when ALT levels are significantly elevated. In some embodiments, serum-detectable CPS1 is indicative of severe ALI. Based on analyzed APAP related human sera, the minimum ALT value to detect serum CPS1 by the immunoblotting assay and particular conditions used (by comparing FIG. 7A and FIG. 8A-C) was 2803 U/L (FIG. 8A). However, depending on conditions and the diagnostic assay used, the precise cut-off value for ALT level to detect serum CPS1 may be significantly altered from that value.

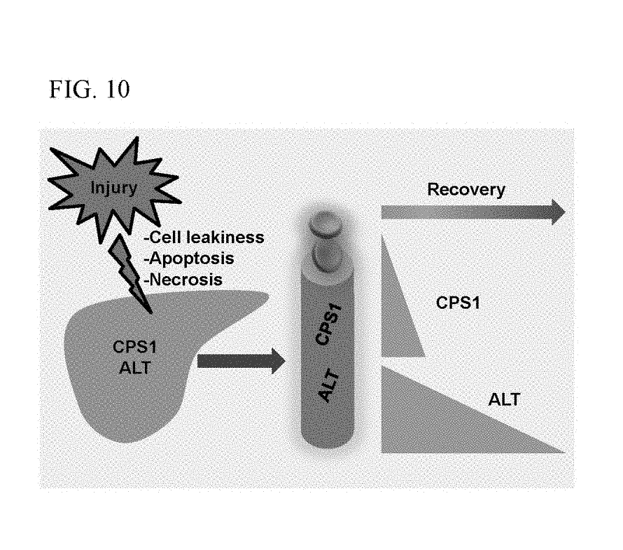

Unexpectedly, it was observed that CPS1 is rapidly eliminated from serum in patients with ALI (FIG. 8). It is contemplated that this elimination represents degradation by serum proteases or uptake by circulating cells such as macrophages; although the present invention is not limited to any particular mechanism of action and an understanding of the mechanism of action is not necessary to practice the present invention. This finding provides a distinct advantage to CPS1 as an early marker of recovery from ALI, since all patients who manifested a rapid drop in CPS1 recovered. One advantage of CPS1 as an ALI biomarkers over other serum biomarkers, such as ALT, is its switch-like disappearance while ALT levels decrease in a more gradual manner (FIG. 10). The half-life of CPS1 was estimated in rats to be 67 minutes (Ozaki et al. Enzyme Protein 48: 213-21, 1994.; herein incorporated by reference in its entirety) and estimated in experiments conducted during development of embodiments of the present invention in mice to be 126 minutes. Taken together, experiments conducted during development of embodiments of the present invention demonstrate CPS1 to be a prognostic biomarker of ALI.

Biomarkers and biomarker panels provided herein are useful for distinguishing samples obtained from individuals with ALI/ALF from samples from individuals without ALI/ALF. In some embodiments, one or more biomarkers are provided for use either alone or in various combinations to detect ALF/ALI, and/or to monitor progression or treatment of ALF/ALI. In some embodiments, CPS1 is detected to identify a subject as having ALI/ALF. In some embodiments, CPS1 is detected as the sole determinant for ALI. In some embodiments, expression levels and/or sample (e.g., serum) concentrations of CPS1 above a threshold level are diagnostic for ALI, in the absence of any other tests and/or indicators. As described in detail herein, exemplary embodiments include the biomarker CPS1 and optionally one or more additional biomarkers of ALF, ALI, chronic liver failure (CLF), liver disease, liver function, liver health, etc. Exemplary markers for use with CPS1 include, but are not limited to: alanine aminotransferase, albumin, aspartate transaminase, alkaline phosphatase, total bilirubin, direct bilirubin, gamma glutamyl transpeptidase, 5′ nucleotidase, serum glucose, lactate dehydrogenase.

While, in some embodiments, CPS1 or one or more of its modified forms (e.g., post-translationally modified products (e.g., reflecting different states of phosphorylation, acetylation, degradation, etc), etc.) are useful alone for providing an ALF/ALI diagnosis and prognosis, methods and kits are also provided herein for grouping CPS1 with additional biomarkers described herein and/or with additional biomarkers not listed herein. In some embodiments, panels of at least two, at least three, at least four, at least five, or at least 6 biomarkers, at least 7 biomarkers, at least 8 biomarkers, at least 9 biomarkers, at least 10 biomarkers, at least 11 biomarkers, at least 12 biomarkers, at least 13 biomarkers, at least 14 biomarkers, at least 15 biomarkers, at least 16 biomarkers, at least 17 biomarkers, at least 18 biomarkers, at least 19 biomarkers, at least 20 biomarkers are provided.

In other embodiments, a CPS1 detection/quantification assay is performed along with one or more additional assays (e.g., liver function tests) in order to diagnose ALI. In some embodiments, a CPS1 detection/quantification assay is performed with a determination of prothrombin time, prothrombin ratio, and/or international normalized ratio. In some embodiments, a positive CPS1 test (e.g., above a threshold) indicates that further testing (e.g., other markers, invasive testing, etc.) should be pursued. In some embodiments, CPS1 is detected as part of a panel of biomarkers that are individually or collectively indicative of and/or diagnostic of ALI. In some embodiments, a biomarker panel comprises 1 (e.g., CPS1), 2, 3, 4, 5, 6, 7, 8, 9, 10, 11, 12, 13, 14, 15, 16, 17, 18, 19, 20 . . . 30 . . . 40, or more biomarkers. In some embodiments, a biomarker panel comprises fewer than 100 biomarkers (e.g., <100, <90, <80, <70, <60, <50, <40, <30, <20, <10, <5).

In some embodiments, the number and identity of biomarkers in a panel are selected based on the sensitivity and specificity for the particular combination of biomarker values. The terms “sensitivity” and “specificity” are used herein with respect to the ability to correctly classify an individual, based on one or more biomarker levels detected in a biological sample, as being having (or continuing to have) ALF/ALI, not having ALF/ALI, or likely to recover from ALF/ALI. “Sensitivity” indicates the performance of the biomarker(s) with respect to correctly classifying individuals having ALF/ALI. “Specificity” indicates the performance of the biomarker(s) with respect to correctly classifying individuals who do not have ALF/ALI. For example, 85% specificity and 90% sensitivity for a panel of markers used to test a set of control samples (such as samples from individuals without ALF/ALI) and test samples (such as samples from individuals with ALF/ALI) indicates that 85% of the control samples were correctly classified as control samples by the panel, and 90% of the test samples were correctly classified as test samples by the panel.

In some embodiments, CPS1 is detected with other biomarkers that are diagnostic of ALI. In some embodiments, such biomarkers are genes and/or their products that are up- or down-regulated upon ALI. In certain embodiments, CPS1 and/or the other biomarkers of the panel distinguish between ALI and other liver conditions, states, or diseases. In certain embodiments, CPS1 and/or the other biomarkers of the panel characterize the severity of ALI. In certain embodiments, the time course profile of CPS1 and/or the other biomarkers of the panel characterize liver disease persistence or a likely change towards liver disease recovery or worsening of liver disease. In certain embodiments, CPS1 and/or the other biomarkers of the panel indicate various treatments (e.g., surgery, no surgery, pharmaceutical treatment, etc.).

In some embodiments, provided herein are methods of assessing and/or detecting ALI in a subject as part of a panel of tests for liver function, disease, and/or injury. In some embodiments, serum CSP1 is detected in combination with a standard liver panel. For example, in some embodiments, serum CPS1 is detected in addition to one or more of: alanine aminotransferase (reference range=10-40 IU/L), albumin (reference range=3.5 to 5.3 g/dL), aspartate transaminase (reference range=6-40 IU/L), alkaline phosphatase (reference range=30 to 120 IU/L), total bilirubin (reference range=0.1-1.0 mg/dL), direct bilirubin (reference range=0.1-0.4 mg/dL), gamma glutamyl transpeptidase (reference range=0-42 IU/L), prothrombin time (PT), prothrombin ratio (PR), international normalized ratio (INR), 5′ nucleotidase, coagulation test, serum glucose, ammonia level, cytokine levels, lactate dehydrogenase, etc. In some embodiments, methods and/or reagents are provided for detecting the presence or level of alanine aminotransferase in a sample from a subject (Berk P, Korenblat K. Approach to the patient with jaundice or abnormal liver tests. In: Goldman L, Schafer A I, eds. Cecil Medicine. 24th ed. Philadelphia, Pa: Saunders Elsevier; 2011:chap 149.; Pratt D S. Liver chemistry and function tests. In: Feldman M, Friedman L S, Brandt L J, eds. Sleisenger and Fordtran's Gastrointestinal and Liver Disease. 9th ed. Philadelphia, Pa: Saunders Elsevier; 2010:chap 73.; herein incorporated by reference in their entireties).

In some embodiments, methods and/or reagents are provided for detecting the presence or level of albumin in a sample from a subject (Berk P, Korenblat K. Approach to the patient with jaundice or abnormal liver tests. In: Goldman L, Schafer A I, eds. Cecil Medicine. 24th ed. Philadelphia, Pa.: Saunders Elsevier; 2011:chap 149.; Pratt D S. Liver chemistry and function tests. In: Feldman M, Friedman L S, Brandt L J, eds. Sleisenger and Fordtran's Gastrointestinal and Liver Disease. 9th ed. Philadelphia, Pa.: Saunders Elsevier; 2010:chap 73.; herein incorporated by reference in their entireties).

In some embodiments, methods and/or reagents are provided for detecting the presence or level of one or more transaminases (e.g., ALT or aspartate transaminase) in a sample from a subject (Fischbach F T, Dunning M B III, eds. (2009). Manual of Laboratory and Diagnostic Tests, 8th ed. Philadelphia: Lippincott Williams and Wilkins.; herein incorporated by reference in its entirety).

In some embodiments, methods and/or reagents are provided for detecting the presence or level of alkaline phosphatase in a sample from a subject (Chernecky C C, Berger B J (2008). Laboratory Tests and Diagnostic Procedures, 5th ed. St. Louis: Saunders.; Fischbach F T, Dunning M B III, eds. (2009). Manual of Laboratory and Diagnostic Tests, 8th ed. Philadelphia: Lippincott Williams and Wilkins.; Pagana K D, Pagana T J (2010). Mosby's Manual of Diagnostic and Laboratory Tests, 4th ed. St. Louis: Mosby Elsevier.; herein incorporated by reference in their entireties).

In some embodiments, methods and/or reagents are provided for detecting the presence or level of total or direct bilirubin in a sample from a subject (Fischbach F T, Dunning M B III, eds. (2009). Manual of Laboratory and Diagnostic Tests, 8th ed. Philadelphia: Lippincott Williams and Wilkins.; Pagana K D, Pagana T J (2010). Mosby's Manual of Diagnostic and Laboratory Tests, 4th ed. St. Louis: Mosby.; herein incorporated by reference in their entireties).

In some embodiments, methods and/or reagents are provided for determining prothrombin time (PT), prothrombin ratio (PR), international normalized ratio (INR) from a sample from a subject (Pagana K D, Pagana T J (2010). Mosby's Manual of Diagnostic and Laboratory Tests, 4th ed. St. Louis: Mosby Elsevier.; Chernecky C C, Berger B J (2008). Laboratory Tests and Diagnostic Procedures, 5th ed. St. Louis: Saunders.; Fischbach F T, Dunning M B III, eds. (2009). Manual of Laboratory and Diagnostic Tests, 8th ed. Philadelphia: Lippincott Williams and Wilkins.; Pagana K D, Pagana T J (2010). Mosby's Manual of Diagnostic and Laboratory Tests, 4th ed. St. Louis: Mosby Elsevier.; herein incorporated by reference in their entireties).

In some embodiments, methods and/or reagents are provided for detecting the presence or level of 5′ nucleosidase in a sample from a subject (Pratt D S. Liver chemistry and function tests. In: In: Feldman M, Friedman L S, Brandt L J, eds. Sleisenger & Fordtran's Gastrointestinal and Liver Disease. 9th ed. Philadelphia, Pa.: Saunders Elsevier; 2010:chap 73.; herein incorporated by reference in its entirety).

In some embodiments, methods and/or reagents are provided for performing coagulation tests in a sample from a subject (Thachil, Postgrad Med J. 2008 April;84(990):177-81.; herein incorporated by reference in its entirety).

In some embodiments, methods and/or reagents are provided for detecting the presence or level of serum glucose in a sample from a subject (American Diabetes Association. Standards of medical care in diabetes—2012. Diabetes Care. 2011 January;35 Suppl 1:S11-63.; Buse J B, Polonsky K S, Burant C F. Type 2 diabetes mellitus. In: Melmed S, Polonsky K S, Larsen P R, Kronenberg H M, Larsen P R, eds. Williams Textbook of Endocrinology. 12th ed. Philadelphia, Pa.: Saunders Elsevier; 2011:chap 31.; Inzucchi S E, Sherwin R S. Type 2 diabetes mellitus. In: Goldman L, Schafer A I, eds. Cecil Medicine. 24th ed. Philadelphia, Pa.: Saunders Elsevier; 2011:chap 237.; herein incorporated by reference in their entireties).

In some embodiments, methods and/or reagents are provided for detecting the presence or level of lactate dehydrogenase in a sample from a subject (Gallagher P G. Hemolytic anemias: red cell membrane and metabolic defects In: Goldman L, Schafer A I, eds. Cecil Medicine. 24th ed. Philadelphia, Pa.: Saunders Elsevier; 2011:chap 164.; Gregg X, Prchal J T. Red Blood Cell Enzymopathies. In: Hoffman R, Benz E J, Shattil S S, et al, eds. Hematology: Basic Principles and Practice. 5th ed. Philadelphia, Pa.: Elsevier Churchill Livingstone; 2008:chap 45.; herein incorporated by reference in their entireties).

In some embodiments, methods comprise contacting a sample or a portion of a sample from a subject with at least one capture reagent, wherein each capture reagent specifically binds a biomarker whose presence and/or level is being detected. In some embodiments, the method comprises contacting the sample, or proteins from the sample, with at least one antibody or aptamer, wherein each antibody/aptamer specifically binds a biomarker whose levels are being detected (e.g., CPS1).

In some embodiments, a method comprises detecting the level of a first biomarker (e.g., CPS1) by contacting a sample with detection and/or capture reagents specific for that biomarker (e.g., antibodies/aptamers for CPS1), and then detection one or more additional biomarkers.

The biomarkers identified herein provide a number of choices for subsets of panels of biomarkers that can be used to effectively identify ALF/ALI. Selection of the appropriate number of such biomarkers (e.g., for use with CPS1) may depend on the specific combination of biomarkers chosen. In addition, in any of the methods described herein, except where explicitly indicated, a panel of biomarkers may comprise additional biomarkers not listed herein. In some embodiments, a method comprises detecting the presence and/or level of CPS1, alone or in addition to at least one biomarker, at least two biomarkers, at least three biomarkers, at least four biomarkers, at least five biomarkers, at least six biomarkers, at least seven biomarkers, at least eight biomarkers, or nine biomarkers of, for example, ALF, ALI, CLF, liver disease, liver function, liver health, etc. Exemplary markers for use with CPS1 include, but are not limited to: alanine aminotransferase, albumin, aspartate transaminase, alkaline phosphatase, total bilirubin, direct bilirubin, gamma glutamyl transpeptidase, 5′ nucleotidase, serum glucose, blood ammonia, lactate dehydrogenase. In some embodiments, a method comprises detecting the level of any number and combination of CPS1 and biomarkers of ALF, ALI, CLF, liver disease, liver function, liver health, etc. (e.g., alanine aminotransferase, albumin, aspartate transaminase, alkaline phosphatase, total bilirubin, direct bilirubin, gamma glutamyl transpeptidase, 5′ nucleotidase, serum glucose, lactate dehydrogenase). In some embodiments, a method comprises detecting the level of CPS1 along with determining a subject's prothrombin time, prothrombin ratio, and/or international normalized ratio.

### Exemplary Uses of Biomarkers

In various exemplary embodiments, methods are provided for determining whether a subject has suffered ALI and/or is suffering from ALF. Methods are also provided for assessing the effectiveness of ALI/ALF treatment. In some embodiments, biomarkers are indicative of comorbidity with viral hepatitis (e.g., types A, B, C, D, E) of other diseases/conditions affecting the liver. In some embodiments, methods comprise detecting the presence of one or more biomarkers (e.g., CPS1). In some embodiments, methods comprise measuring the level or concentrations of one or more biomarkers (e.g., CPS1) by any number of analytical methods, including any of the analytical methods described herein. These biomarkers are, for example, present at different levels in ALF-positive and ALF-negative subjects (e.g., present in ALF⁺ subjects and absent in ALF⁻ subjects). In some embodiments, detection of the differential levels of a biomarker in an individual can be used, for example, to permit the determination of whether the individual has ALF, has suffered ALI, etc.). In some embodiments, detection of the presence of a biomarker (e.g., CPS1) in an individual can be used, for example, to permit the determination that the individual has ALF. In some embodiments, any of the biomarkers described herein may be used to monitor ALF in an individual over time, and to permit the determination of whether treatment is effective or whether prognosis is improving or deteriorating.

In addition to testing biomarker levels as a stand-alone diagnostic test, in some embodiments, biomarker levels are tested in conjunction with other markers or assays indicative of ALF (e.g., imaging, biopsy, symptom analysis, prothrombin time, prothrombin ratio, international normalized ration, etc.). In addition to testing biomarker levels in conjunction with other ALF diagnostic methods, information regarding the biomarkers can also be evaluated in conjunction with other types of data, particularly data that indicates an individual's risk for ALF (e.g., lifestyle, genetics, age, etc.). These various data can be assessed by automated methods, such as a computer program/software, which can be embodied in a computer or other apparatus/device.

### Detection of Biomarkers and Determination of Biomarker Levels

The presence of a biomarker (e.g., CS1) or a biomarker level for the biomarkers described herein can be detected using any of a variety of analytical methods. In one embodiment, a biomarker level is detected using a capture reagent. In various embodiments, the capture reagent is exposed to the biomarker in solution or is exposed to the biomarker while the capture reagent is immobilized on a solid support. In other embodiments, the capture reagent contains a feature that is reactive with a secondary feature on a solid support. In these embodiments, the capture reagent is exposed to the biomarker in solution, and then the feature on the capture reagent is used in conjunction with the secondary feature on the solid support to immobilize the biomarker on the solid support. The capture reagent is selected based on the type of analysis to be conducted. Capture reagents include but are not limited to aptamers, antibodies, adnectins, ankyrins, other antibody mimetics and other protein scaffolds, autoantibodies, chimeras, small molecules, F(ab′)2 fragments, single chain antibody fragments, Fv fragments, single chain Fv fragments, nucleic acids, lectins, ligand-binding receptors, affybodies, nanobodies, imprinted polymers, avimers, peptidomimetics, hormone receptors, cytokine receptors, and synthetic receptors, and modifications and fragments of these.

In some embodiments, biomarker presence or level is detected using a biomarker/capture reagent complex. In some embodiments, the biomarker presence or level is derived from the biomarker/capture reagent complex and is detected indirectly, such as, for example, as a result of a reaction that is subsequent to the biomarker/capture reagent interaction, but is dependent on the formation of the biomarker/capture reagent complex.

In some embodiments, biomarker presence or level is detected directly from the biomarker in a biological sample.

In some embodiments, biomarkers are detected using a multiplexed format that allows for the simultaneous detection of two or more biomarkers in a biological sample. In some embodiments of the multiplexed format, capture reagents are immobilized, directly or indirectly, covalently or non-covalently, in discrete locations on a solid support. In some embodiments, a multiplexed format uses discrete solid supports where each solid support has a unique capture reagent associated with that solid support, such as, for example quantum dots. In some embodiments, an individual device is used for the detection of each one of multiple biomarkers to be detected in a biological sample. Individual devices are configured to permit each biomarker in the biological sample to be processed simultaneously. For example, a microtiter plate can be used such that each well in the plate is used to analyze one or more of multiple biomarkers to be detected in a biological sample.

In one or more of the foregoing embodiments, a fluorescent tag is used to label a component of the biomarker/capture reagent complex to enable the detection of the biomarker level. In various embodiments, the fluorescent label is conjugated to a capture reagent specific to any of the biomarkers described herein using known techniques, and the fluorescent label is then used to detect the corresponding biomarker level. Suitable fluorescent labels include rare earth chelates, fluorescein and its derivatives, rhodamine and its derivatives, dansyl, allophycocyanin, PBXL-3, Qdot 605, Lissamine, phycoerythrin, Texas Red, and other such compounds.

In some embodiments, the fluorescent label is a fluorescent dye molecule. In some embodiments, the fluorescent dye molecule includes at least one substituted indolium ring system in which the substituent on the 3-carbon of the indolium ring contains a chemically reactive group or a conjugated substance. In some embodiments, the dye molecule includes an AlexFluor molecule, such as, for example, AlexaFluor 488, AlexaFluor 532, AlexaFluor 647, AlexaFluor 680, or AlexaFluor 700. In some embodiments, the dye molecule includes a first type and a second type of dye molecule, such as, e.g., two different AlexaFluor molecules. In some embodiments, the dye molecule includes a first type and a second type of dye molecule, and the two dye molecules have different emission spectra.

Fluorescence can be measured with a variety of instrumentation compatible with a wide range of assay formats. For example, spectrofluorimeters have been designed to analyze microtiter plates, microscope slides, printed arrays, cuvettes, etc. See Principles of Fluorescence Spectroscopy, by J. R. Lakowicz, Springer Science +Business Media, Inc., 2004. See Bioluminescence & Chemiluminescence: Progress & Current Applications; Philip E. Stanley and Larry J. Kricka editors, World Scientific Publishing Company, January 2002.

In one or more embodiments, a chemiluminescence tag is optionally used to label a component of the biomarker/capture complex to enable the detection of a biomarker level. Suitable chemiluminescent materials include any of oxalyl chloride, Rodamin 6G, Ru(bipy)32+, TMAE (tetrakis(dimethylamino)ethylene), Pyrogallol (1,2,3-trihydroxibenzene), Lucigenin, peroxyoxalates, Aryl oxalates, Acridinium esters, dioxetanes, and others.

In some embodiments, the detection method includes an enzyme/substrate combination that generates a detectable signal that corresponds to the biomarker level (e.g., using the techniques of ELISA, Western blotting, isoelectric focusing). Generally, the enzyme catalyzes a chemical alteration of the chromogenic substrate which can be measured using various techniques, including spectrophotometry, fluorescence, and chemiluminescence. Suitable enzymes include, for example, luciferases, luciferin, malate dehydrogenase, urease, horseradish peroxidase (HRPO), alkaline phosphatase, beta-galactosidase, glucoamylase, lysozyme, glucose oxidase, galactose oxidase, and glucose-6-phosphate dehydrogenase, uricase, xanthine oxidase, lactoperoxidase, microperoxidase, and the like.

In some embodiments, the detection method is a combination of fluorescence, chemiluminescence, radionuclide or enzyme/substrate combinations that generate a measurable signal. In some embodiments, multimodal signaling has unique and advantageous characteristics in biomarker assay formats.

In some embodiments, the biomarker levels for the biomarkers described herein is detected using any analytical methods including, singleplex aptamer assays, multiplexed aptamer assays, singleplex or multiplexed immunoassays, mRNA expression profiling, miRNA expression profiling, mass spectrometric analysis, histological/cytological methods, etc. as discussed below.

Determination of Biomarker Levels using Immunoassays

Immunoassay methods are based on the reaction of an antibody to its corresponding target or analyte and can detect the analyte in a sample depending on the specific assay format. To improve specificity and sensitivity of an assay method based on immuno-reactivity, monoclonal antibodies and fragments thereof are often used because of their specific epitope recognition. Polyclonal antibodies have also been successfully used in various immunoassays because of their increased affinity for the target as compared to monoclonal antibodies. Immunoassays have been designed for use with a wide range of biological sample matrices. Immunoassay formats have been designed to provide qualitative, semi-quantitative, and quantitative results.

Quantitative results are generated through the use of a standard curve created with known concentrations of the specific analyte to be detected. The response or signal from an unknown sample is plotted onto the standard curve, and a quantity or level corresponding to the target in the unknown sample is established.

Numerous immunoassay formats have been designed. ELISA or EIA can be quantitative for the detection of an analyte. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition; herein incorporated by reference in its entirety).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or for quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Determination of Biomarker Levels Using Gene Expression Profiling

Measuring mRNA in a biological sample may, in some embodiments, be used as a surrogate for detection of the level of a corresponding protein in the biological sample. Thus, in some embodiments, a biomarker or biomarker panel described herein can be detected by detecting the appropriate RNA.

In some embodiments, mRNA expression levels are measured by reverse transcription quantitative polymerase chain reaction (RT-PCR followed with qPCR). RT-PCR is used to create a cDNA from the mRNA. The cDNA may be used in a qPCR assay to produce fluorescence as the DNA amplification process progresses. By comparison to a standard curve, qPCR can produce an absolute measurement such as number of copies of mRNA per cell. Northern blots, microarrays, RNAseq, Invader assays, and RT-PCR combined with capillary electrophoresis have all been used to measure expression levels of mRNA in a sample. See Gene Expression Profiling: Methods and Protocols, Richard A. Shimkets, editor, Humana Press, 2004; herein incorporated by reference in its entirety.

### Detection of Biomarkers Using In Vivo Molecular Imaging Technologies

In some embodiments, a biomarker described herein may be used in molecular imaging tests. For example, an imaging agent can be coupled to a capture reagent, which can be used to detect the biomarker in vivo.

In vivo imaging technologies provide non-invasive methods for determining the state of a particular disease in the body of an individual. For example, entire portions of the body, or even the entire body, may be viewed as a three dimensional image, thereby providing valuable information concerning morphology and structures in the body. Such technologies may be combined with the detection of the biomarkers described herein to provide information concerning the biomarker in vivo.

Advances in the use of in vivo molecular imaging technologies include the development of new contrast agents or labels, such as radiolabels and/or fluorescent labels, which can provide strong signals within the body; and the development of powerful new imaging technology, which can detect and analyze these signals from outside the body, with sufficient sensitivity and accuracy to provide useful information. The contrast agent can be visualized in an appropriate imaging system, thereby providing an image of the portion or portions of the body in which the contrast agent is located. The contrast agent may be bound to or associated with a capture reagent, with a peptide or protein, or an oligonucleotide (for example, for the detection of gene expression), or a complex containing any of these with one or more macromolecules and/or other particulate forms.

The contrast agent may also feature a radioactive atom that is useful in imaging. Suitable radioactive atoms include technetium-99m or iodine-123 for scintigraphic studies. Other readily detectable moieties include, for example, spin labels for magnetic resonance imaging (MRI) such as, for example, iodine-123 again, iodine-131, indium-111, fluorine-19, carbon-13, nitrogen-15, oxygen-17, gadolinium, manganese or iron. Such labels are well known in the art and could easily be selected by one of ordinary skill in the art.

Standard imaging techniques include but are not limited to magnetic resonance imaging, computed tomography scanning, positron emission tomography (PET), single photon emission computed tomography (SPECT), and the like. For diagnostic in vivo imaging, the type of detection instrument available is a major factor in selecting a given contrast agent, such as a given radionuclide and the particular biomarker that it is used to target (protein, mRNA, and the like). The radionuclide chosen typically has a type of decay that is detectable by a given type of instrument. Also, when selecting a radionuclide for in vivo diagnosis, its half-life should be long enough to enable detection at the time of maximum uptake by the target tissue but short enough that deleterious radiation of the host is minimized.

Exemplary imaging techniques include but are not limited to PET and SPECT, which are imaging techniques in which a radionuclide is synthetically or locally administered to an individual. The subsequent uptake of the radiotracer is measured over time and used to obtain information about the targeted tissue and the biomarker. Because of the high-energy (gamma-ray) emissions of the specific isotopes employed and the sensitivity and sophistication of the instruments used to detect them, the two-dimensional distribution of radioactivity may be inferred from outside of the body.

Commonly used positron-emitting nuclides in PET include, for example, carbon-11, nitrogen-13, oxygen-15, and fluorine-18. Isotopes that decay by electron capture and/or gamma-emission are used in SPECT and include, for example iodine-123 and technetium-99m. An exemplary method for labeling amino acids with technetium-99m is the reduction of pertechnetate ion in the presence of a chelating precursor to form the labile technetium-99m-precursor complex, which, in turn, reacts with the metal binding group of a bifunctionally modified chemotactic peptide to form a technetium-99m-chemotactic peptide conjugate.

Antibodies are frequently used for such in vivo imaging diagnostic methods. The preparation and use of antibodies for in vivo diagnosis is well known in the art. Similarly, aptamers may be used for such in vivo imaging diagnostic methods. The label used will be selected in accordance with the imaging modality to be used, as previously described. In some embodiments, imaging has unique and advantageous characteristics relating to tissue penetration, tissue distribution, kinetics, elimination, potency, and selectivity as compared to other imaging agents.

Such techniques may also optionally be performed with labeled oligonucleotides, for example, for detection of gene expression through imaging with antisense oligonucleotides. These methods are used for in situ hybridization, for example, with fluorescent molecules or radionuclides as the label. Other methods for detection of gene expression include, for example, detection of the activity of a reporter gene.

Another general type of imaging technology is optical imaging, in which fluorescent signals within the subject are detected by an optical device that is external to the subject. These signals may be due to actual fluorescence and/or to bioluminescence. Improvements in the sensitivity of optical detection devices have increased the usefulness of optical imaging for in vivo diagnostic assays.

Other techniques are review, for example, in N. Blow, Nature Methods, 6, 465-469, 2009; herein incorporated by reference in its entirety.

### Determination of Biomarkers Using Histology/Cytology Methods

In some embodiments, the biomarkers described herein may be detected in a variety of tissue samples using histological or cytological methods. In some embodiments, one or more capture reagent/s specific to the corresponding biomarker/s are used in a cytological evaluation of a sample and may include one or more of the following: collecting a cell sample, fixing the cell sample, dehydrating, clearing, immobilizing the cell sample on a microscope slide, permeabilizing the cell sample, treating for analyte retrieval, staining, destaining, washing, blocking, and reacting with one or more capture reagent/s in a buffered solution. In another embodiment, the cell sample is produced from a cell block.

In some embodiments, one or more capture reagent/s specific to the corresponding biomarkers are used in a histological evaluation of a tissue sample and may include one or more of the following: collecting a tissue specimen, fixing the tissue sample, dehydrating, clearing, immobilizing the tissue sample on a microscope slide, permeabilizing the tissue sample, treating for analyte retrieval, staining, destaining, washing, blocking, rehydrating, and reacting with capture reagent/s in a buffered solution. In another embodiment, fixing and dehydrating are replaced with freezing.

### Determination of Biomarker Levels Using Mass Spectrometry Methods

A variety of configurations of mass spectrometers can be used to detect biomarker levels, and to also define posttranslational modifications (e.g., phosphorylation, glycosylation, acetylation) of the biomarker. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al. Anal. Chem. 70:647 R-716R (1998); Kinter and Sherman, New York (2000)).

Protein biomarkers and biomarker levels can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS)N, atmospheric pressure photoionization mass spectrometry (APPI-MS), APPI-MS/MS, and APPI-(MS)N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker levels. Labeling methods include but are not limited to isobaric tag for relative and absolute quantitation (iTRAQ) and stable isotope labeling with amino acids in cell culture (SILAC). In some embodiments, capture reagents are used to selectively enrich samples for candidate biomarker prior to mass spectroscopic analysis.

The foregoing assays enable the detection of biomarker levels that are useful in the methods described herein, where the methods comprise detecting, in a biological sample from an individual, at least one (e.g., CPS1 and/or its post-translationally-modified products), at least two, at least three, at least four, at least five, at least six, at least seven, at least eight, or at least nine biomarkers selected from the described herein or elsewhere. Thus, while some of the described biomarkers may be useful alone for detecting ALF, methods are also described herein for the grouping of multiple biomarkers and subsets of the biomarkers to form panels of two or more biomarkers. In accordance with any of the methods described herein, biomarker levels can be detected and classified individually or they can be detected and classified collectively, as for example in a multiplex assay format.

### Classification of Biomarkers and Calculation of Disease Scores

In some embodiments, a biomarker “signature” for a given diagnostic test contains one or more biomarkers (e.g., a set of markers), each marker having characteristic levels in the populations of interest. Characteristic levels, in some embodiments, may refer to the mean or average of the biomarker levels for the individuals in a particular group. In some embodiments, a diagnostic method described herein can be used to assign an unknown sample from an individual into one of two or more groups: suffered from ALI, having ALF, healthy, etc. The assignment of a sample into one of two or more groups is known as classification, and the procedure used to accomplish this assignment is known as a classifier or a classification method. Classification methods may also be referred to as scoring methods. There are many classification methods that can be used to construct a diagnostic classifier from a set of biomarker levels. In some instances, classification methods are performed using supervised learning techniques in which a data set is collected using samples obtained from individuals within two (or more, for multiple classification states) distinct groups one wishes to distinguish. Since the class (group or population) to which each sample belongs is known in advance for each sample, the classification method can be trained to give the desired classification response. It is also possible to use unsupervised learning techniques to produce a diagnostic classifier.

Common approaches for developing diagnostic classifiers include decision trees; bagging+boosting+forests; rule inference based learning; Parzen Windows; linear models; logistic; neural network methods; unsupervised clustering; K-means; hierarchical ascending/descending; semi-supervised learning; prototype methods; nearest neighbor; kernel density estimation; support vector machines; hidden Markov models; Boltzmann Learning; and classifiers may be combined either simply or in ways which minimize particular objective functions. For a review, see, e.g., Pattern Classification, R. O. Duda, et al., editors, John Wiley & Sons, 2nd edition, 2001; see also, The Elements of Statistical Learning—Data Mining, Inference, and Prediction, T. Hastie, et al., editors, Springer Science+Business Media, LLC, 2nd edition, 2009.

Exemplary embodiments use any number of the biomarkers provided herein in various combinations (e.g., with other biomarkers of ALF, ALI, CLF, liver health, etc.) to produce diagnostic tests for detecting ALI/ALF in a sample from an individual. The markers provided herein can be combined in many ways to produce classifiers. For example, a classifier may comprise CPS1 and ALT. Other exemplary classifiers comprise any suitable combinations of CPS1 and alanine aminotransferase, albumin, aspartate transaminase, alkaline phosphatase, total bilirubin, direct bilirubin, gamma glutamyl transpeptidase, prothrombin time, prothrombin ratio, international normalized ratio, 5′ nucleotidase, serum glucose, lactate dehydrogenase.

In some embodiments, once a panel is defined to include a particular set of biomarkers and a classifier is constructed from a set of training data, the diagnostic test parameters are complete. In some embodiments, a biological sample is run in one or more assays to produce the relevant quantitative biomarker levels used for classification. The measured biomarker levels are used as input for the classification method that outputs a classification and an optional score for the sample that reflects the confidence of the class assignment.

### Kits

Any combination of the biomarkers described herein can be detected using a suitable kit, such as for use in performing the methods disclosed herein. The biomarkers described herein may be combined in any suitable combination (e.g., CPS1 and one or more additional markers), or may be combined with other markers not described herein (e.g., markers of liver health, hepatitis infection, or non-liver markers). Furthermore, any kit can contain one or more detectable labels as described herein, such as a fluorescent moiety, etc.

In some embodiments, a kit includes (a) one or more capture reagents for detecting one or more biomarkers in a biological sample, and optionally (b) one or more software or computer program products for predicting whether the individual from whom the biological sample was obtained is ALF/ALI positive. Alternatively, rather than one or more computer program products, one or more instructions for manually performing the above steps by a human can be provided.

In some embodiments, a kit comprises a solid support, a capture reagent, and a signal generating material. The kit can also include instructions for using the devices and reagents, handling the sample, and analyzing the data. Further the kit may be used with a computer system or software to analyze and report the result of the analysis of the biological sample.

The kits can also contain one or more reagents (e.g., solubilization buffers, detergents, washes, or buffers) for processing a biological sample. Any of the kits described herein can also include, e.g., buffers, blocking agents, mass spectrometry matrix materials, serum/plasma separators, antibody capture agents, positive control samples, negative control samples, software and information such as protocols, guidance and reference data.

In some embodiments, kits are provided for the analysis of ALI/ALF, wherein the kits comprise PCR primers for one or more biomarkers described herein. In some embodiments, a kit may further include instructions for use and correlation of the biomarkers ALI/ALF. In some embodiments, a kit may include a DNA array containing the complement of one or more of the biomarkers described herein, reagents, and/or enzymes for amplifying or isolating sample DNA. The kits may include reagents for real-time PCR, for example, TaqMan probes and/or primers, and enzymes.

For example, a kit can comprise (a) reagents comprising at least one capture reagent for determining the level of one or more biomarkers in a test sample, and optionally (b) one or more algorithms or computer programs for performing the steps of comparing the amount of each biomarker quantified in the test sample to one or more predetermined cutoffs. In some embodiments, an algorithm or computer program assigns a score for each biomarker quantified based on said comparison and, in some embodiments, combines the assigned scores for each biomarker quantified to obtain a total score. Further, in some embodiments, an algorithm or computer program compares the total score with a predetermined score, and uses the comparison to determine likelihood of ALI/ALF. Alternatively, rather than one or more algorithms or computer programs, one or more instructions for manually performing the above steps by a human can be provided.

### Computer Methods and Software

Once a biomarker or biomarker panel is selected, a method for detecting ALI/ALF in an individual may comprise the following: 1) collect or otherwise obtain a biological sample; 2) perform an analytical method to detect and measure the biomarker or biomarkers in the panel in the biological sample; and 3) report the results of the biomarker levels. In some embodiments, the results of the biomarker levels are reported qualitatively rather than quantitatively, such as, for example, a proposed diagnosis or simply a positive/negative result where “positive” and “negative” are defined. In some embodiments, a method for detecting ALI/ALF in an individual may comprise the following: 1) collect or otherwise obtain a biological sample; 2) perform an analytical method to detect and measure the biomarker or biomarkers in the panel in the biological sample; 3) perform any data normalization or standardization; 4) calculate each biomarker level; and 5) report the results of the biomarker levels. In some embodiments, the biomarker levels are combined in some way and a single value for the combined biomarker levels is reported. In this approach, in some embodiments, the reported value may be a single number determined from the sum of all the marker calculations that is compared to a pre—set threshold value that is an indication of the presence or absence of disease. Or the diagnostic score may be a series of bars that each represent a biomarker value and the pattern of the responses may be compared to a pre-set pattern for determination of the presence or absence of disease.

At least some embodiments of the methods described herein can be implemented with the use of a computer. Such a computer may comprise various connected (e.g., directly, wirelessly, over a network, over the web, etc.) or integrated components, including but not limited to: a processor, input device, output device, storage device, computer-readable storage media reader, communications system, processing acceleration (e.g., DSP or special-purpose processors), memory, etc. In some embodiments, computer-readable storage media reader is further coupled to computer-readable storage media, the combination comprehensively representing remote, local, fixed and/or removable storage devices plus storage media, memory, etc. for temporarily and/or more permanently containing computer-readable information, which can include storage device, memory and/or any other such accessible system resource. A system may also comprise software elements including an operating system and other code, such as programs, data and the like.

In one aspect, the system comprises a database containing features of biomarkers characteristic of ALI/AFI. The biomarker data (or biomarker information) can be utilized as an input to the computer for use as part of a computer implemented method. The biomarker data can include the data as described herein.

The system may be connectable to a network to which a network server and one or more clients are connected. The network may be a local area network (LAN) or a wide area network (WAN), as is known in the art. Preferably, the server includes the hardware necessary for running computer program products (e.g., software) to access database data for processing user requests.

The system may include one or more devices that comprise a graphical display interface comprising interface elements such as buttons, pull down menus, scroll bars, fields for entering text, and the like as are routinely found in graphical user interfaces known in the art. Requests entered on a user interface can be transmitted to an application program in the system for formatting to search for relevant information in one or more of the system databases. Requests or queries entered by a user may be constructed in any suitable database language. The graphical user interface may be generated by a graphical user interface code as part of the operating system and can be used to input data and/or to display inputted data. The result of processed data can be displayed in the interface, printed on a printer in communication with the system, saved in a memory device, and/or transmitted over the network or can be provided in the form of the computer readable medium.

The system can be in communication with an input device for providing data regarding data elements to the system (e.g., expression values). In one aspect, the input device can include a gene expression profiling system including, e.g., a mass spectrometer, gene chip or array reader, and the like.

The methods and apparatus for analyzing biomarker information according to various embodiments may be implemented in any suitable manner, for example, using a computer program operating on a computer system. A conventional computer system comprising a processor and a random access memory, such as a remotely-accessible application server, network server, personal computer or workstation may be used. Additional computer system components may include memory devices or information storage systems, such as a mass storage system and a user interface, for example a conventional monitor, keyboard and tracking device. The computer system may be a stand-alone system or part of a network of computers including a server and one or more databases.

The biomarker analysis system can provide functions and operations to complete data analysis, such as data gathering, processing, analysis, reporting and/or diagnosis. For example, in one embodiment, the computer system can execute the computer program that may receive, store, search, analyze, and report information relating to the biomarkers. The computer program may comprise multiple modules performing various functions or operations, such as a processing module for processing raw data and generating supplemental data and an analysis module for analyzing raw data and supplemental data to generate a disease status and/or diagnosis. Detecting ALI/ALF in a subject may comprise generating or collecting any other information, including additional biomedical information, regarding the condition of the individual relative to the disease, identifying whether further tests may be desirable, or otherwise evaluating the health status of the individual.

While various embodiments have been described as methods or apparatuses, it should be understood that embodiments can be implemented through code coupled with a computer, e.g., code resident on a computer or accessible by the computer. For example, software and databases could be utilized to implement many of the methods discussed above. Thus, in addition to embodiments accomplished by hardware, it is also noted that these embodiments can be accomplished through the use of an article of manufacture comprised of a computer usable medium having a computer readable program code embodied therein, which causes the enablement of the functions disclosed in this description. Therefore, it is desired that embodiments also be considered protected by this patent in their program code means as well. Furthermore, the embodiments may be embodied as code stored in a computer-readable memory of virtually any kind including, without limitation, RAM, ROM, magnetic media, optical media, or magneto-optical media. Even more generally, the embodiments could be implemented in software, or in hardware, or any combination thereof including, but not limited to, software running on a general purpose processor, microcode, programmable logic arrays (PLAs), or application-specific integrated circuits (ASICs).

### Methods of Treatment

In some embodiments, following a determination that a subject has suffered ALI or suffers from ALF, the subject is appropriately treated. In some embodiments, therapy is administered to treat ALF. In some embodiments, therapy is administered to prevent the ongoing cause of ALI (e.g., remove the toxin causing the liver injury, treat the disease that is the source of the ALI). In some embodiments, therapy is administered to treat complications of ALI/ALF, for example, relieving excess fluid in the brain, preventing or treating bleeding, treating the encephalopathy, etc. In some embodiments, treatment comprises liver transplant.

In some embodiments, following a determination that a subject has suffered ALI or suffers from ALF, the subject is tested for one or more other disease states (e.g., infection, hepatitis, etc.)

In some embodiments, methods of monitoring ALI/ALF and/or treatment of ALI/ALF are provided. In some embodiments, the present methods of detecting ALI/ALF are carried out at a time 0. In some embodiments, the method is carried out again at a time 1, and optionally, a time 2, and optionally, a time 3, etc., in order to monitor the progression of ALI/ALF or to monitor the effectiveness of one or more treatments of ALI/ALF. Time points for detection may be separated by, for example at least 4 hours, at least 8 hours, at least 12 hours, at least 1 day, at least 2 days, at least 4 days, at least 1 week, at least 2 weeks, at least 1 month, at least 2 months, at least 3 months, at least 4 months, at least 6 months, or by 1 year or more. In some embodiments, a treatment regimen is altered based upon the results of monitoring (e.g., upon determining that a first treatment is ineffective). In some embodiments, the level of intervention (e.g., monitoring in an intensive care unit with 1:1 nursing-to-patient care ratio; monitoring in an intensive care unit with 1:4 nursing-to-paiten care ratio; or monitoring in a route hospital bed without continuous cardiac/respiratory monitoring) may be altered.

### EXPERIMENTAL

### Example 1

### Martials and Methods

### Isolation and Treatment of Primary Mouse Hepatocytes.

Primary hepatocytes from mice were isolated (Snider et al. J Cell Biol 195: 217-29, 2011.; herein incorporated by reference in its entirety). The liver was perfused with 3-5 ml of perfusion media through the portal vein with a flow rate of 3 ml/min followed by perfusion with 15-20 ml of digestion media containing 150 units/ml of collagenase-II (Worthington) at the same flow rate. After the first wash, the cell pellet was suspended in 6 ml of ice-cold wash-media and separated on ice-cold percoll gradient (SIGMA, 15% in PBS pH 7.5, 500 rpm for 10 min) to remove dead cells. The cell pellet was washed with ice-cold wash-media and suspended in culture media (William's medium E supplemented with 10% FBS and 1% penicillin-streptomycin, 37° C.) and plated [0.20×106 cells/ml on collagen-I coated 6-well plates (BD BioCoat)] for media analysis and biochemical experiments. For immunostaining, cells were plated on 4-well collagen-I coated chamber slides (BD BioCoat). After 1 h (37° C., 5% CO₂) to allow attachment, the cell culture media was replaced (to remove any debris or unattached cells) with fresh media and allowed to equilibrate for an additional 12 h (37° C., 5% CO₂). The cultured hepatocytes were then treated with FasL (0.5 μg/ml, BD Pharmingen), acetaminophen (APAP) (1 Sigma) or hypoosmotic media (200 mOsm/L) in the absence of fetal bovine serum. FasL administration, liver tissue and blood analysis in mice. For in vivo experiments, FVB non-transgenic mice were fasted overnight followed by intraperitoneal administration of FasL (0.15 μg/g), APAP (700 mg/Kg) or vehicle (saline or DMSO respectively). After 4 h (FasL injected) or 8 h (APAP injected), mice were sacrificed by CO2 inhalation and livers were isolated and divided into pieces that were stored in liquid nitrogen (for biochemical analysis) or fixed in 10% formalin (for hematoxylin and eosin staining and histological analysis). Blood samples were collected from the mice by intra-cardiac puncture and stored overnight (4° C.) before enzyme or biochemical analysis. Serum ALT levels were determined using a Vetscan-vs2 instrument (ABAXIS) using the comprehensive diagnostic profile cartridge or using Unit for Laboratory Animal Medicine (ULAM) core facility at University of Michigan. All mice received humane care and animal use was performed in accordance with a protocol that was approved by the Committee for the Use and Care of Animals at the University of Michigan.

### Preparation of Liver and Hepatocyte Lysates, and Biochemical Analysis.

Total liver or primary hepatocyte lysates were prepared by homogenizing liver tissue or cells using 2× Tris-glycine SDS sample buffer. Sera were also mixed with 2× SDS-containing sample buffer before analysis. Proteins were separated using SDS-polyacrylamide gel electrophoresis (SDS-PAGE), then stained with Coomassie blue or transferred to polyvinylidene fluoride membranes followed by blotting with antibodies to CPS1 (Abcam), active caspases 3 and 7 (Cell signaling), high mobility group box 1 protein (HMGB1) (Abcam); actin (Lab Vision); keratin 18 (Ku et al. Methods Cell Biol 78: 489-517, 2004.) and keratin 19 (Toivola et al. J Cell Biol 164: 911-921, 2004.; herein incorporated by reference in its entirety).

### Immunofluorescence Staining and Confocal Microscopy Imaging.

Immunostaining of hepatocytes or liver tissue sections were carried out as described in Ku et al. Methods Cell Biol 78: 489-517, 2004; herein incorporated by reference in its entirety. Hepatocytes or liver sections were fixed with methanol (10 min, −20° C.) and air dried (1 h). Nonspecific binding was blocked by incubation in blocking buffer [phosphate buffered saline (PBS) with 2.5% wt/vol bovine serum albumin and 2% goat serum] for 10 min. For hepatocyte staining, anti-CPS1 antibody was incubated in blocking buffer for 1 h at 22° C. and for immunostaining of liver sections, CPS1 antibody was co-incubated overnight (−4° C.) along with the antibodies to keratin 18 or keratin 19. After three 5-min washes in PBS, slides were incubated with Alexa Fluor—conjugated secondary antibody (30 min). The slides were washed three times (5-min/wash) in PBS, air dried and mounted in ProLong Gold containing DAPI (Invitrogen). Cells were imaged using a laser-scanning confocal microscope (FluoView 500; Olympus) with a 60× oil immersion (1.4 NA) objective.

Analysis of the Media from Cultured Hepatocyte.

After individual treatment of the hepatocytes, culture plates were centrifuged (1200 rpm, 5 min) followed by collection of the culture media without disturbing the cells. The collected media was repelleted (1200 rpm, 5 min) and the supernatant was concentrated using Centricon YM-10 filters [(Millipore) (typically, 2 ml was concentrated to 0.1 ml)] then mixed with 2× SDS-PAGE sample buffer for subsequent immunoblotting.

### Human Serum Samples.

Serum samples were obtained from patients hospitalized at the University of Michigan Medical center with the diagnosis of acute liver injury (ALI) and enrolled in the Acute Liver Failure Study Group Registry. Blood samples were collected daily during hospitalization. Serum was also analyzed from patients with chronic hepatitis B and C who attend the Liver Clinic at the University of Michigan. In addition, available randomly selected unused serum was obtained from the Hospital Chemistry Laboratory from individuals who had normal serum ALT levels and was classified as ‘Control Serum’. No donor identifiers were collected and the underlying reason for blood collection from these individuals is not known. Serum collection and analysis was carried out under protocols approved by the University of Michigan Human Subjects Committee.

### Mass Spectrometry.

Coomassie stained gel bands were excised, destained, and digested with trypsin overnight. The peptides were extracted from the gel bands, ziptipped (C18 cleanup), spotted on the MALDI target, and analyzed by MALDI TOF/TOF. Peptides were identified searching UniProt mouse data base utilizing Mascot v2.2 search tool.

### Statistical Analysis.

Statistical analyses were performed using ANOVA (for cell count experiment), unpaired t-test with Welch's correction (for time dependent CPS1 and ALT turnover experiment) and Column statistics (to determine half-life) using GraphPad Prism 5 statistical software.

### Ethics Approvals.

The approval to conduct the human and animal studies was obtained from the University of Michigan committees for human and animal use, respectively.

### Example 2

### CPS1 is a Major Protein Released to Culture Media During Apoptotic Hepatocyte Injury

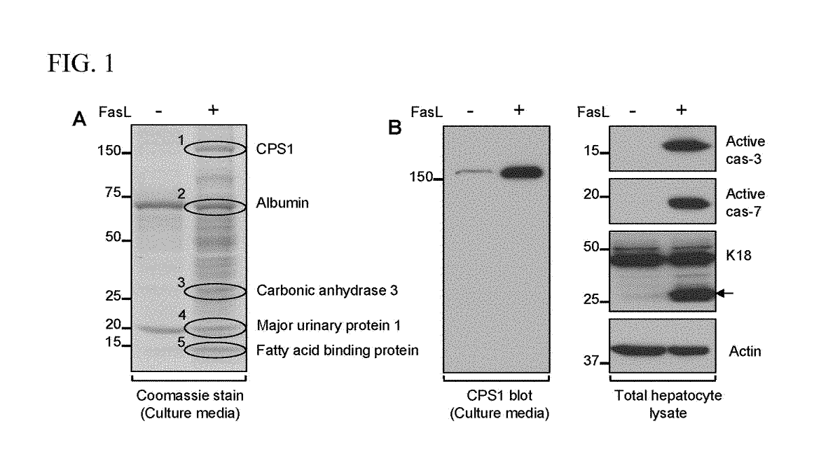

To identify potential new biomarkers of liver cell death and injury, primary hepatocytes were isolated from mice and treated with FasL (0.5 μg/ml) to induce apoptosis. After 6 h, the cell culture media was collected, concentrated, separated by SDS-PAGE and stained with Coomassie blue dye. Release of many protein bands was observed in the culture media collected from FasL-treated as compared to untreated hepatocytes (FIG. 1A). The most intense bands (labeled 1-5, FIG. 1A) were subjected to mass spectrometry analysis with the identity of the bands included in FIG. 1A. Based on the protein identification, CPS1 for further detailed study because of its high abundance and selective expression in the liver, unique localization in mitochondria and, because it had not been reported as a biomarker for apoptotic cell. Mass spectrometry results were confirmed by immunoblotting the concentrated hepatocyte culture media with an antibody specific to CPS1 (FIG. 1B, left panel). It was demonstrated that CPS1 release occurs in association with the predicted FasL-mediated apoptosis as evident by caspases 3 and 7 activation and generation of the caspase-cleaved keratin 18 apoptotic fragment (FIG. 1B, right panel, band highlighted by arrow). These findings indicate that primary hepatocytes release CPS1 during apoptotic mouse hepatocellular injury. The extracellular released CPS1 is readily visualized by Coomassie stain and is as abundant as the released albumin (FIG. 1A).

### Example 3

### Mouse Primary Hepatocytes and Intact Liver Release CPS1 During Different Types of Cell Injury

CPS1 release in response to FasL was compared with other different types of hepatocyte injury that do not involve apoptosis per se. For this, isolated mouse primary hepatocytes were challenged with hypoosmotic stress (200 mOsm/L; selected as a form of mechanical stress), APAP (selected as a form of necrotic stress) or FasL. Analysis of the concentrated culture media showed a Coomassie stain that is at the predicted molecular size of CPS1 (nearly 160 kDa), which was confirmed by immunoblotting using anti-CPS1 antibody (FIG. 2A). APAP treatment of hepatocytes results in cell death via necrosis, which is confirmed by absence of caspase activation and release of the nuclear protein high mobility group box 1 protein (HMGB1) (FIG. 2A). Notably, CPS1 is released during hypoosmotic conditions without any evidence for necrosis or apoptosis (FIG. 2A) indicating that the hepatocytes become leaky, although the present invention is not limited to any particular mechanism of action and an understanding of the mechanism of action is not necessary to practice the present invention. The extent of hepatocyte death after each treatment by trypan blue staining after trypsinization. In the absence of HMGB1 release or caspase activation, hypoosmotic treatment showed no detectable cell death (FIG. 2B), however, marked CPS1 release occurred (FIG. 2A). In agreement with the increased CPS1 release, FasL and APAP treatment showed significant cell death (FIG. 2B). Immune staining further verified the release of CPS1 from hepatocytes in response to the different challenges shown in FIG. 2C. Untreated control hepatocytes showed punctuated, dense and a uniformly distributed CPS1 throughout the cytoplasm (FIG. 2C). All three challenges resulted in loss of cytoplasmic CPS1 staining (FIG. 2C, arrows). Collectively these data suggest that CPS1 release occurs in response to different modes of hepatocyte death and injury including hypoosmotic stress, apoptosis and necrosis.

Selective expression of CPS1 was examined in hepatocyte and ductal cells, the most abundant two cell types in the liver, by immunostaining. As shown in FIG. 3A (panel c), CPS1 co-localize with hepatocyte specific marker, keratin 18 (Omary et al. Hepatology 35: 251-257. 2002.; herein incorporated by reference in its entirety). Ductal cells do not express CPS1 as evidenced by the absence of co-localization with ductal cell specific maker, keratin 19 (FIG. 3B (panel c); Toivola et al. J Cell Biol 164: 911-921, 2004.; herein incorporated by reference in its entirety). These results indicate that CPS1 is selectively expressed in hepatocytes in the liver.

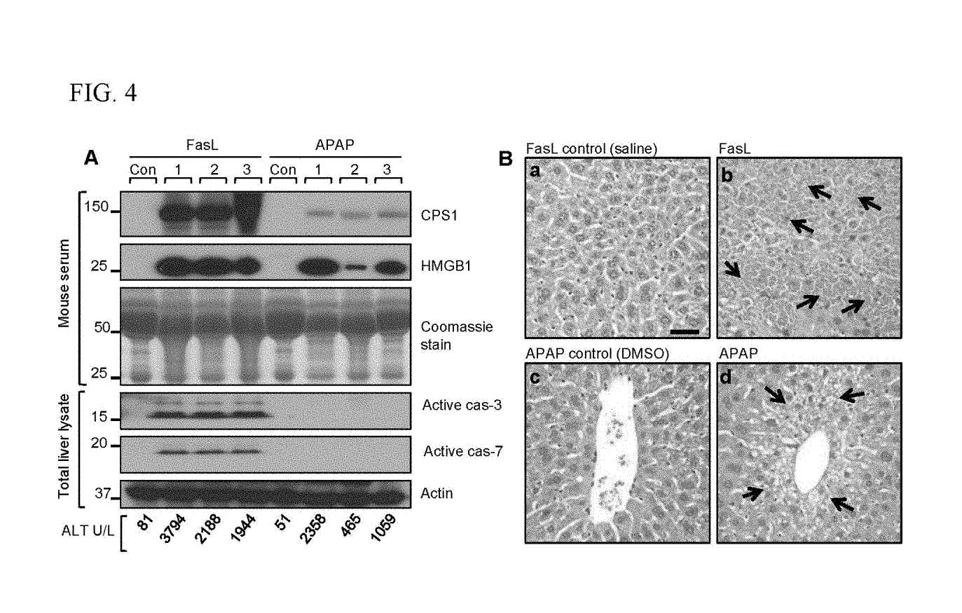

Experiments were conducted during development of embodiments of the present invention to demonstrate that CPS1 is detectable in mouse serum in response to apoptotic and necrotic liver injury. Mice were injected with FasL in saline (to induce apoptosis), or with APAP in DMSO (to induced necrosis) or were injected with vehicle (saline or DMSO). The serum and liver histology were then analyzed. For both the APAP and FasL conditions, CPS1 was more easily detected in the serum of the mice and parallels the elevation of ALT (FIG. 4A). Activation of caspases in the total liver lysates confirms the apoptosis in FasL treated mice but not in the APAP intoxicated mice (FIG. 4A). Histological analysis of liver sections showed marked liver hemorrhage in mice treated with FasL (FIG. 4B, upper right, arrows) and necrotic cell death in mice treated with APAP (FIG. 4B, lower right, arrows) as compared with control livers. The observed low amount of CPS1 and HMGB1 release in APAP intoxicated mice compared to FasL treated mice (FIG. 4A) reflects the extent of liver damage caused by each model (note that FasL treated mice were very sick compared to APAP intoxicated mice by the time of sacrifice). This is also confirmed by the relative serum ALT levels (FIG. 4A). The same interpretation applies to the ex vivo conditions, whereby the higher levels of CPS1 release in APAP treated primary hepatocytes (FIG. 2A) compared to FasL and hypoosmosis is due to the extent of hepatocyte damage and the selected dosage for each experimental condition.

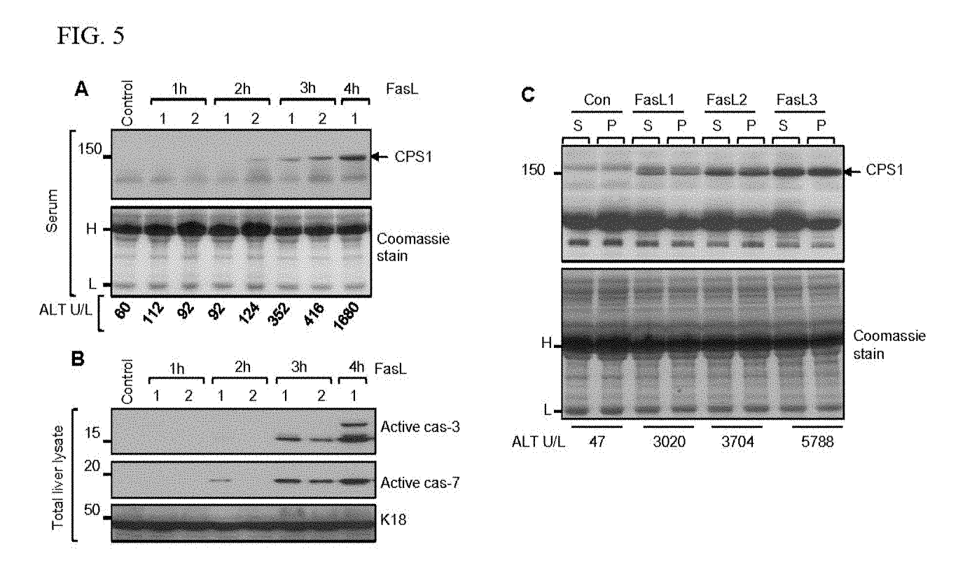

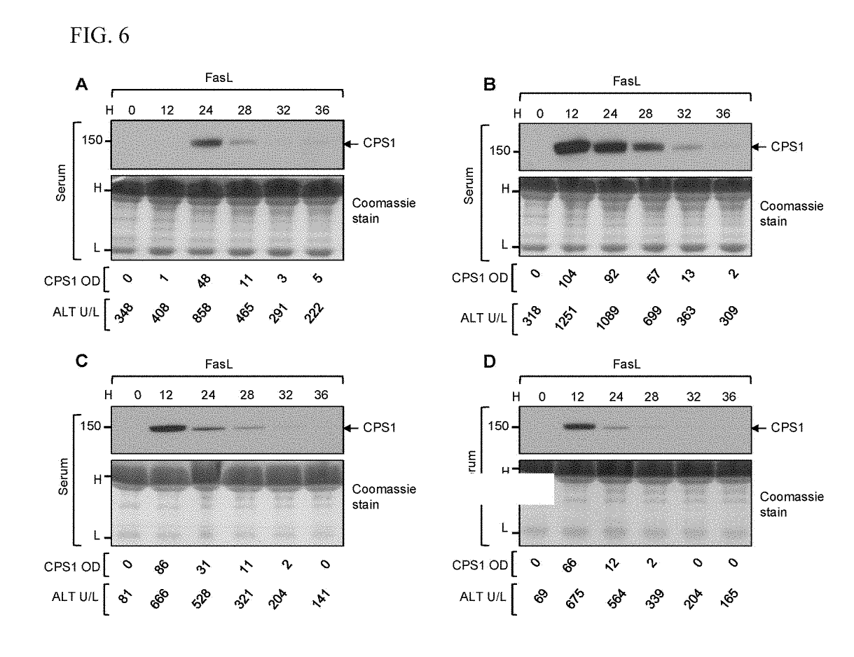

Time course analysis demonstrates that CPS1 is readily detected in serum as early as 2 h after mouse apoptotic liver injury (FIG. 5A) with parallel activation of caspases (FIG. 5B), thereby indicating that CPS1 is released during apoptotic hepatocyte injury. Both serum and plasma are equally effective in detecting CPS1 in the blood after FasL-induced liver injury (FIG. 5C). Collectively, these data clearly indicate that CPS1 can be detected in serum during apoptotic and necrotic liver injury in mice. Further, the approximate half-life (combination of turnover and ongoing release) of CPS1 in mouse serum in response to FasL treatment (0.075 μg/g) is 126 minutes (FIG. 6).

### Example 4

### CPS1 is Present in Human Sera of Patients with Acute Liver Injury

Experiments were conducted during development of embodiments of the present invention to evaluate detecting CPS1 in human sera from patients suffering from ALI due to APAP, ischemia or Wilson disease. Serial daily serum samples (from day one of hospitalization) were obtained and analyzed for CPS1 by immunoblotting. For APAP, serum CPS1 was readily detectable at day 1 then decreased at day 2 and became completely undetectable from days 3-5 (FIG. 7A). Notably, while CPS1 was undetectable in this patient at days 3-5, serum ALT levels remained significantly elevated (FIG. 7A). In the case of ALI related to ischemia, the serum CPS1 was elevated on day 1 (FIG. 7B) and also became undetectable while serum ALT remained very high. Both patients recovered from their ALI without the need for liver transplantation. In contrast, the patient with Wilson disease resulted in a rapid rise in serum CPS1 and ALT on day 6 (FIG. 7C) and subsequently died from complications related to ALI. These data indicate that serum CPS1 is readily detectable in sera of human patients with ALI, while serum ALT levels are also very high, but CPS1 level drop precipitously as ALI resolves, while ALT values remain elevated.

### Example 5

### Serum CPS1 Turns Over Rapidly in Patients with APAP-related ALF

Experiments were conducted during development of embodiments of the present invention to explore the rapid drop in serum CPS1 detected in patients with APAP and ischemia (FIG. 7A, B) and compared this to changes in serum ALT. Serial serum samples from additional patients with APAP-related ALI were analyzed. As shown in FIG. 8A-C, serum CPS1 was readily detectable on day 1 of hospitalization when serum ALT is significantly elevated. Consistently, CPS1 disappeared rapidly upon treatment of ALI, while serum ALT levels remained elevated. Densitometric analyses of CPS1 immunoblots were performed and the percent remaining intensity of CPS1 (relative to Day 1) was compared with the percent remaining ALT values. As shown in FIG. 8D, serum CPS1 rapidly decreased by 90% of Day 1 values by day 3, while serum ALT levels only decreased by 55% (all patients recovered). This significant difference in serum CPS1 versus ALT levels maintained up to day 4 (FIG. 7D) without signs of diminishing. Data indicates that CPS1 is a sensitive prognostic marker for recovery in cases of ALI.

### Example 6

### Serum CPS1 in Mouse and Human Chronic Liver Diseases

Experiments were conducted during development of embodiments of the present invention to detect serum CPS1 in chronic liver injury in mice and humans. To test the CPS1 release during chronic mouse liver injury, mice were fed 0.1% DDC (a porphyrinogenic hepatotoxin) for 10-days, 15-days, 6-weeks and 3-months and serum CPS1 levels were analyzed. Serum CPS1 levels were highest at the earlier and more acute period of liver injury (10 days), which correlated in general terms with the ALT levels (FIG. 9A). Control mice serum showed no detectable CPS1, and there were no detectable levels of the necrosis marker HMGB1 at any of the time points tested (FIG. 9A). Histologic assessment of the livers showed early evidence of cell drop off at days 10 and 15 with subsequent stabilization as injury becomes chronic (FIG. 9B).

Experiments were conducted during development of embodiments of the present invention to detect CPS1 in the sera of patients with chronic hepatitis B and C, as compared with serum from controls who had normal ALT. In contrast to DDC induced mouse chronic liver injury, there was no detectable CPS1 in any of the serum sample of patients with hepatitis B or C (FIG. 9C, D). In these patients, serum ALT varied but was elevated in most of the cases (FIG. 9C, D). As expected, sera from the controls also showed no detectable serum CPS1. Consistent with the mouse data, there were no serum HMGB1 in any of the patient serum samples (FIG. 9C, D). These data indicate that serum CPS1 is not a useful marker for chronic human viral hepatitis.

### REFERENCES

The following references, as well as any patents or publications recited above, are herein incorporated by reference in their entireties.

1. Blei A T. Selection for acute liver failure: have we got it right? Liver Transpl 11: 30-4, 2005.

2. Butler S L, Dong H, Cardona D, Jia M, Zheng R, Zhu H, Crawford J M, Liu C. The antigen for Hep Par 1 antibody is the urea cycle enzyme carbamoyl phosphate synthatase 1. Lab Invest 88: 78-88, 2008.

3. Crouser E D, Julian M W, Huff J E, Struck J, Cook C H. Carbamoyl phosphate synthase-1: A marker of mitochondrial damage and depletion in the liver during sepsis. Crit Care Med 34: 2439-46, 2006.

4. Greystoke A, Dean E, Saunders M P, Cummings J, Hughes A, Ranson M, Dive C, Renehan A G. Multi-level evidence that circulating CK18 is a biomarker of tumor burden in colorectal cancer. Br J Cancer 107: 1518-24. 2012.

5. Glinghammar B, Rafter I, Lindström A K, Hedberg J J, Andersson H B, Lindblom P, Berg A L, Cotgreave I. Detection of the mitochondrial and catalytically active alanine aminotransferase in human tissue and plasma. Int J Mol Med 23: 621-31, 2009.

6. Hu Z, Lausted C, Yoo H, Yan X, Brightman A, Chen J, Wang W, Bu X, Hood L. Quantitative liver-specific protein fingerprint in blood: a signature for hepatotoxicity. Theranostics 4: 215-228, 2014.

7. Joka D, Wahl K, Moeller S, Schlue J, Vaske B, Bahr M J, Manns M P, Schulze-Osthoff K, Bantel H. Prospective biopsy-controlled evaluation of cell death biomarkers for prediction of liver fibrosis and nonalcoholic steatohepatitis. Hepatology 55: 455-64, 2012.

8. Ku N O, Toivola D M, Zhou Q, Tao G Z, Zhong B, Omary M B. Studying simple epithelial keratins in cells and tissues. Methods Cell Biol 78: 489-517, 2004.

9. Lindblom P, Rafter I, Copley C, Andersson U, Hedberg J J, Berg AL, Samuelsson A, Hellmold H, Cotgreave I, Glinghammar B. Isoforms of alanine aminotransferases in human tissue and serum-Differential tissue expression using novel antibodies. Arch Biochem Biophys 466: 66-77, 2007.

10. Malhi H, Guicciardi M E, Gores G J. Hepatocyte Death: A Clear and Present Danger. Physiol Rev 90: 1165-94, 2010.

11. Martinez A I, Pérez-Arellano I, Pekkala S, Barcelona B, Cervera J. Genetic, structural and biochemical basis of carbamoyl phosphate synthetase 1 Deficiency. Mol Genet Metab 101: 311-23, 2010.

12. McPhail M J, Wendon J A, Bernal W. Meta-analysis of performance of Kings's College Hospital Criteria in prediction of outcome in non-paracetamol-induced acute liver failure. J Hepatol 53: 492-9. 2010.

13. Neill M A, Aschner J, Barr F, Summar M L. Quantitative RT-PCR comparison of the urea and nitric oxide cycle gene transcripts in adult human tissues. Mol Genet Metab 97: 121-7, 2009.

14. Omary M B, Ku N-O, Toivola D M. Keratins: Guardians of the liver. Hepatology 35: 251-257. 2002.

15. Omary M B, Ku N O, Strnad P, Hanada S. Toward unraveling the complexity of simple epithelial keratins in human disease. J Clin Invest 119: 1794-805, 2009.

16. Oyama K, Fushida S, Kinoshita J, Okamoto K, Makino I, Nakamura K, Hayashi H, Inokuchi M, Nakagawara H, Tajima H, Fujita H, Takamura H, Ninomiya I, Kitagawa H, Fujimura T, Ohta T. Serum cytokeratin 18 as a biomarker for gastric cancer. Clin Exp Med July 24, Epub ahead of print. 2012.

17. Ozaki M, Terada K, Kanazawa M, Fujiyama S, Tomita K, Mori M. Enzyme-linked immunosorbent assay of carbamoyl phosphate synthetase 1: Plasma enzyme in rat experimental hepatitis and its clearance. Enzyme Protein 48: 213-21, 1994.

18. Rapp B, Häberle J, Linnebank M, Wermuth B, Marquardt T, Harms E, Koch H G. Genetic analysis of carbamoylphosphate synthetase I and ornithine transcarbamylase deficiency using fibroblasts. Eur J Pediatr 160: 283-7, 2001.

19. Rutherford A, King L Y, Hynan L S, Vedvyas C, Lin W, Lee W M, Chung R T; ALF Study Group. Development of an accurate index for predicting outcomes of patients with acute liver failure. Gastroenterology 143: 1237-43, 2012.

20. Snider N T, Weerasinghe S V, Singla A, Leonard J M, Hanada S, Andrews P C, Lok A S, Omary M B. Energy determinants GAPDH and NDPK act as genetic modifiers for hepatocyte inclusion formation. J Cell Biol 195: 217-29, 2011.

21. Struck J, Uhlein M, Morgenthaler N G, Fürst W, Höflich C, Bahrami S, Bergmann A, Volk H D, Redl H. Release of the mitochondrial enzyme carbamoyl phosphate synthase 1 under septic condition. Shock 23: 533-8, 2005.

22. Tas F, Karabulut S, Bilgin E, Sen F, Yildiz I, Tastekin D, Ciftci R, Duranyildiz D. Clinical significance of serum M30 and M65 levels in metastatic pancreatic adenocarcinoma. Tumour Biol June 21, Epub ahead of print. 2013.

23. Toivola D M, Krishnan S, Binder H J, Singh H K, Omary M B. Keratins modulate colonocyte electrolyte transport via protein mistargeting. J Cell Biol 164: 911-921, 2004.

24. Unuma K, Aki T, Matsuda S, Funakoshi T, Yoshida K, Uemura K. Elimination and active extrusion of liver mitochondrial proteins during lipopolysaccharide administration in rat. Hepatol Res 43: 526-34, 2013.

1. A method of detecting acute liver failure (ALF) or acute liver injury (ALI) in a subject, comprising detecting the level of carbamoyl phosphate synthatase-1 (CPS1) and/or one of its post-translationally modified forms in a sample from the subject, wherein the subject is identified as having ALF if the level of CPS1 is above a threshold level.

2. The method of claim 1, further comprising detecting and/or determining one or more of alanine aminotransferase (ALT), albumin, aspartate transaminase, alkaline phosphatase, total bilirubin, direct bilirubin, gamma glutamyl transpeptidase, 5′ nucleotidase, serum glucose, lactate dehydrogenase.

3. The method of claim 2, further comprising detecting ALT, wherein the subject is identified as having ALF or ALI if the levels at least one of CPS1 and ALT is above a threshold level for the respective biomarker.

4. The method of claim 3, further comprising detecting alanine aminotransferase (ALT), wherein the subject is identified as having ALF or ALI if the levels both CPS1 and ALT is above a threshold range for the respective biomarker.

5. The method of any one of claim 1-4, comprising detecting three or more biomarkers.

6. The method of claim 5, comprising detecting ten or more biomarkers.

7. The method of any one of claims 1-6, comprising detecting 100 or fewer biomarkers.

8. The method of claim 1, wherein the sample is a blood product selected from whole blood; plasma; serum; and filtered, concentrated, fractionated or diluted samples of the preceding.

9. The method of claim 1, wherein the method comprises contacting biomarkers of the sample from the subject with a set of biomarker capture reagents, wherein each biomarker capture reagent of the set of biomarker capture reagents specifically binds to a different biomarker being detected.

10. The method of claim 9, wherein each biomarker capture reagent is an antibody.

11. The method of claim 1, wherein the method further comprises treating the subject for ALF or ALI.

13. The method of claim 1, wherein the method further comprises performing one or more additional tests of liver health.

14. The method of claim 13, wherein said tests for liver health are selected from imaging, biopsy, and additional blood tests.

15. A method of monitoring progression of ALF or ALI in a subject, comprising detecting the level of carbamoyl phosphate synthatase-1 (CPS1) in a sample from the subject at a first time point, and measuring the level of CPS1 at a second time point, wherein ALF or ALI are decreasing in severity if the level of CPS1 is nearer to a control value at the second time or subsequent point than at the first or prior time points.

16. The method of claim 15, wherein ALF or ALI are increasing in severity if the level of CPS1 is further from a control value at the second or subsequent time points than at the first time point or prior time points, and

17. The method of claim 15, further comprising detecting and/or determining one or more of alanine aminotransferase (ALT), albumin, aspartate transaminase, alkaline phosphatase, total bilirubin, direct bilirubin, gamma glutamyl transpeptidase, 5′ nucleotidase, serum glucose, lactate dehydrogenase at the first, second and subsequent time points.

18. The method of claim 17, further comprising detecting ALT level at the first and subsequent timepoints, wherein ALF or ALI are decreasing in severity if the level of CPS1 is nearer to a control value at the second time point than at the first time point, irrespective of the ALT level.

19. A method of evaluating ALF or ALI in a subject, comprising:
(a) detecting levels of carbamoyl phosphate synthatase-1 (CPS1) alanine aminotransferase (ALT) in a sample from the subject at a first time point, wherein elevated levels of CPS1 and ALT at the first time point is diagnostic for ALI;
(b) detecting levels of CPS1 at subsequent time points, wherein progressive decrease in CPS1 level indicated a decrease in ALF or ALI severity, and wherein CPS1 level below a threshold value indicates likely recovery from ALF or ALI.

20. A method of monitoring treatment and prognosis of acute liver failure (ALF) or ALI in a subject, comprising:
(a) detecting the level of CPS1 in a sample from the subject at a first time point;
(b) administering a treatment for ALF to the subject; and
(c) detecting the level of CPS1 in a sample from the subject at subsequent time points; wherein the treatment is effective or recovery is likely if the CPS1 level is nearer to a control value at the second time point than at the first time point.

21. Reagents for the detection of ALF or ALI comprising capture reagents for the detection of CPS1 and reagents for the detection of one or more of alanine aminotransferase (ALT), albumin, aspartate transaminase, alkaline phosphatase, bilirubin, gamma glutamyl transpeptidase, 5′ nucleotidase, serum glucose, and lactate dehydrogenase.

22. The reagents of claim 21, wherein the capture reagents comprise antibodies.

23. A method of detecting hepatocyte mitochondrial damage that may reflect leaky or dying hepatocytes in a subject, comprising:
(a) detecting the level of carbamoyl phosphate synthatase-1 (CPS1) and/or one of its post-translationally modified forms in a sample from the subject;
(b) detecting the level of alanine aminotransferase (ALT) in the sample from the subject;
(c) identifying said subject as having hepatocyte mitochondrial damage that may reflect leaky or dying hepatocytes if the levels of CPS1 and ALT are above threshold levels.
